(19) United States
(12) Patent Application Publication
(10) Pub. No.: US 2018/0356413 A1
(43) Pub. Date: Dec. 13, 2018

(54) Diagnostic Test with Lateral Flow Test Strip

(71) Applicant: MOLOGIC LIMITED, Thurleigh, Bedfordshire (GB)

(72) Inventor: Gita Parekh, Wootton, Bedfordshire (GB)

(73) Assignee: MOLOGIC LIMITED, Thurleigh, Bedfordshire (GB)

(21) Appl. No.: 15/778,163

(22) PCT Filed: Nov. 23, 2016
(86) PCT No.: PCT/GB2016/053684

§ 371 Date: May 22, 2018

(30) Foreign Application Priority Data
Nov. 23, 2015 (GB) 1520657.6

(51) Int. Cl. G01N 33/543; G01N 33/573; G01N 33/68; B01L 3/00; A61M 1/28

(52) U.S. Cl. CPC G01N 33/54306; G01N 33/573; G01N 33/6869; B01L 3/5023; A61M 1/287; G01N 2800/06; A61M 2210/12; G01N 2333/5412; B01L 2200/026; B01L 2300/0825; B01L 2400/0406; A61M 2210/0625; A61M 2210/1017; G01N 2333/96494

(57) ABSTRACT

A diagnostic test apparatus comprises:
an elongate housing defining a test strip holder containing a lateral flow test strip;
a fluid sampling chamber provided with an opening connecting the fluid sampling chamber with the test strip holder;
a viewing window in the elongate housing allowing reading of one or more portions of the lateral flow test strip; and
a connector configured to couple the diagnostic test apparatus to a bulk source of fluid.

The apparatus may be included in a kit of parts. The apparatus is useful for detecting peritonitis. The bulk source of fluid may be peritoneal dialysate. Various markers may be determined in the bulk source of fluid such as MMP8, IL-6, HNE, MMP2, MMP9, TIMP1, TIMP2, NGAL, A1AT, desmosine, fibrinogen, IL-8, calprotectin, fMLP, IL1b, cystatin C, HSA, RBP4, SPD, MPO, sICAM and TNFa. Detection of the markers to indicate peritonitis may also inform treatment choices.

### FIELD OF THE DISCLOSURE

The present disclosure relates to a diagnostic test apparatus and an associated kit of parts. It further relates to methods of testing fluids. In particular, it discloses a diagnostic test apparatus which may be used for testing a fluid. The present diagnostic test apparatus may find particular application in the testing of bulk sources of fluid. An example application is the testing of peritoneal dialysate for infections, e.g. peritonitis.

### BACKGROUND TO THE DISCLOSURE

Peritoneal Dialysis (PD) is the preferred management method for kidney failure patients in the home environment, enabling patients to maintain some semblance of a normal life and regular employment. PD is a passive activity, requiring multiple (typically 4) bag changes a day to remove toxins from the blood stream. PD is an alternative to haemodialysis and involves the use of the patient's peritoneum in the abdomen as an exchange membrane for exchanging substances with the blood. A catheter is surgically implanted in the patient permitting fluid access to the peritoneal cavity. Fluid is then flushed into and out of the abdomen either at night during sleep, known as Automated Peritoneal Dialysis, or continuously, known as Continuous Ambulatory Peritoneal Dialysis.

The PD procedure is shown schematically in FIG. 1, which illustrates a patient 1, catheter 2, dialysis fluid bag 3 containing the fluid to be passed into the abdominal cavity 4 via feed line 5 and the dialysate fluid bag 6 containing the waste fluid withdrawn from the abdominal cavity 4 via a drain line 7. PD involves relatively large volumes of fluid. Each exchange of fluid into and out of the abdomen may by up to 2.5 litres or more. Thus the dialysate bag 6 may be of a large capacity—typically 0.5 to 2.5 litres or more. For convenience and hygiene the fluid drained from the abdominal cavity is passed directly to the dialysate bag 6 via the drain line 7.

Unfortunately, infection is the primary cause for patients to lose the freedom of PD, as it causes damage to the peritoneum and a loss of the infusion line/catheter. Costs of treatment are high and, where the line is lost, surgical intervention is required.

### SUMMARY OF THE DISCLOSURE

Catching an infection early would prevent damage and line loss yet there is no way for a patient to detect an emergent infection other than a change in fluid colour or aroma at discharge. The methods and devices of the present disclosure integrate with PD fluid-handling equipment, in particular the bags and associated tubing. In some embodiments, the methods and devices interface with the waste tube of a PD fluid bag and test the waste fluid as it is discarded. The user will be able to observe a simple visual result and take corrective action long before an infection takes hold. The patient will be able to remain at home, reducing healthcare costs and extending the clinical utility of PD.

The methods and devices of the present disclosure provide reliable detection of the first measurable response of the innate immune system, namely neutrophil infiltration.

In a first aspect there is provided a diagnostic test apparatus comprising:
an elongate housing defining a test strip holder containing a lateral flow test strip;
a fluid sampling chamber provided with an opening connecting the fluid sampling chamber with the test strip holder;
a viewing window in the elongate housing allowing reading of one or more portions of the lateral flow test strip; and
a connector configured to couple the diagnostic test apparatus to a bulk source of fluid.

The diagnostic test apparatus advantageously allows for the testing of fluid directly as it is fed into, drained from or contained in a bulk source of fluid such as a dialysate fluid bag. This allows the patient to carry out the test without unhygienic exposure to the waste fluid. In addition, the test may be carried out while emptying the dialysate fluid bag to waste via a drain tube.

The diagnostic test apparatus may also retain a fluid sample from the bulk source of fluid during the test. This may allow the fluid sample to be conveniently retained for later testing, re-testing or analysis of the fluid sample at a remote site if needed. This is particularly advantageous where the test is carried out during draining to waste since otherwise no fluid would be obtainable for re-testing.

The connector may be configured to establish fluid communication between the bulk source of fluid and the fluid sampling chamber.

The connector may comprise an interference fit connector, an adhesive connector, or a clip connector.

The connector may comprise a tubular element. The tubular element may contain the fluid sampling chamber. The tubular element may comprise a first end and a second end and the fluid sampling chamber may be interposed between the first end and the second end.

In one example both the first end and the second end are open to thereby enable a flowing stream of fluid to pass through the fluid sampling chamber during testing. This is particularly beneficial for testing while draining a fluid to waste along a tube or while filling a container along a tube.

In another example the first end is open and the second end is closed to thereby enable a volume of fluid to pass into, and be retained within, the fluid sampling chamber during testing. This is particularly beneficial for testing a source of fluid retained in a closed container where it is desirable to prevent leakage of fluid.

The tubular element may be configured for push-fit coupling to a tube of the bulk source of fluid. The tubular element may comprise an O-ring seal for engaging the tube of the bulk source of fluid.

Alternatively, the connector may comprise a clip for clipping to a tube of the bulk source of fluid.

Alternatively, the tubular element may comprise an adhesive element for adhering to a surface of a container of the bulk source of fluid. The adhesive element may comprise an adhesive pad provided on an end of the tubular element.

The connector may be integral with the elongate housing.

The diagnostic test apparatus may further comprise a puncturing element for opening fluid communication between the bulk source of fluid and the fluid sampling chamber.

The puncturing element may be contained within the fluid sampling chamber.

The puncturing element may puncture an element of the bulk source of fluid during coupling of the connector to the bulk source of fluid. Alternatively, the puncturing element may puncture an element of the bulk source of fluid after coupling of the connector to the bulk source of fluid.

The puncturing element may comprises a piercing element. The piercing element may be statically-mounted within the connector or movably-mounted within the connector to be movable from a retracted position to an extended position. The piercing element may be manually movable into the extended position by operation of a twistable and/or pushable plunger. The puncturing element may comprise a pin, tube or blade.

The diagnostic test apparatus may further comprise a wick element for wicking fluid from the fluid sampling chamber into the test strip holder.

The wick element may project through the opening into the fluid sampling chamber.

The wick element may comprise an end portion of the lateral flow test strip. Alternatively, the wick element may comprise a separate wick element that is in fluid contact with the lateral flow test strip.

The wick element comprises an elongate wick or a tubular or part-tubular element.

The wick may comprises a foam element.

The fluid sampling chamber may be located at a first end of the elongate housing.

The fluid sampling chamber may be formed integrally with the elongate housing.

In a further aspect of the present disclosure there is provided a kit of parts comprising:
a diagnostic test apparatus as described above; and
a bulk source of fluid.

The bulk source of fluid may comprise a container holding the fluid and the connector of the diagnostic test apparatus may be configured to be coupled to the container.

The container may be closed prior to coupling of the diagnostic test apparatus.

The container may comprise a storage bag and one or more tubes extending from the storage bag.

The connector of the diagnostic test apparatus may be configured to be coupled to the storage bag, for example to be coupled to the one or more tubes.

The container may hold greater than 100 ml, optionally greater than 250 ml, optionally greater than 1000 ml of fluid.

The container may be a dialysate bag, optionally a peritoneal dialysate bag.

In a further aspect of the present disclosure there is provided a method of carrying out a diagnostic test on a bulk source of fluid using a diagnostic test apparatus of the type comprising:
an elongate housing defining a test strip holder containing a lateral flow test strip, a fluid sampling chamber provided with an opening connecting the fluid sampling chamber with the test strip holder, and a viewing window in the elongate housing allowing reading of one or more portions of the lateral flow test strip;
   the method comprising the steps of:
coupling the diagnostic test apparatus to the bulk source of fluid by use of a connector;
during or after coupling of the connector, establishing a fluid communication between the bulk source of fluid and the fluid sampling chamber of the diagnostic test apparatus to convey fluid from the bulk source of fluid into the fluid sampling chamber;
causing fluid to pass from the fluid sampling chamber through the opening to wet the lateral flow test strip.

The fluid may be wicked from the fluid sampling chamber through the opening.

The connector may be interference fit, adhered, or clipped to the bulk source of fluid.

Fluid may be conveyed from the bulk source of fluid through the fluid sampling chamber as a flowing stream of fluid that passes through the fluid sampling chamber during testing. Alternatively, fluid may be conveyed from the bulk source of fluid into, and retained within, the fluid sampling chamber during testing.

The fluid communication between the bulk source of fluid and the fluid sampling chamber of the diagnostic test apparatus may be established by puncturing an element of the bulk source of fluid.

The fluid communication between the bulk source of fluid and the fluid sampling chamber of the diagnostic test apparatus may be maintained for a period of time to sufficiently saturate the test strip as would be readily understood by one skilled in the art. This may be a period of around 1-10 seconds, such as around 5 seconds. An indicator of wetting of the test strip may be included to confirm to the user that sufficient sample has reached the viewing window. This may be a colour change on the test strip for example. Once this indicator has been detected, the diagnostic test apparatus may then be disconnected from the bulk source of fluid.

The bulk source of fluid may be punctured during coupling of the connector to the bulk source of fluid. Alternatively, the bulk source of fluid may be punctured after coupling of the connector to the bulk source of fluid.

The element of the bulk source of fluid may be punctured by driving the element against a puncturing element of the connector. Alternatively, the element of the bulk source of fluid may be punctured by driving a puncturing element of the connector through the element of the bulk source of fluid.

The bulk source of fluid may comprise a container holding the fluid and the connector of the diagnostic test apparatus may be configured to be coupled to the container.

The container may be closed prior to coupling of the diagnostic test apparatus.

The container may be a dialysate bag, optionally a peritoneal dialysate bag.

The bulk source of fluid may comprise a continuous flow of fluid along a drain line and the connector of the diagnostic test apparatus may be configured to be coupled to the drain line.

In a further aspect the present disclosure provides a method of detecting peritonitis in a subject comprising determining the level of at least one marker selected from matrix metalloprotease 8 (MMP8), human neutrophil elastase (HNE), MMP2, MMP9, tissue inhibitor of metalloproteinase 1 (TIMP1), TIMP2, neutrophil gelatinase-associated lipocalin (NGAL), alpha-1 antitrypsin (A1AT), desmosine, fibrinogen, interleukin-6 (IL-6), IL-8, calprotectin, N-Formylmethionyl-leucyl-phenylalanine (fMLP), interleukin-1beta (ID1b), cystatin C, human serum albumin (HAS), retinol binding protein 4 (RBP4), surfactant protein D (SPD), myeloperoxidase (MPO), soluble intercellular adhesion molecule (sICAM) and tumour necrosis factor alpha (TNFa) in a sample of peritoneal dialysate wherein an increased level of at least one of MMP8, HNE, MMP2, MMP9, TIMP1, TIMP2, NGAL, A1AT, desmosine, IL-6, IL-8, calprotectin, fMLP, IL1b, cystatin C, HSA, RBP4, SPD, MPO, sICAM and TNFa and/or a decreased level of fibrinogen is indicative of peritonitis.

According to the invention, the sample is a peritoneal dialysate. Thus, the methods are performed as in vitro methods using the isolated sample, which can for example be retrieved from a dialysate fluid bag.

Peritonitis is defined as inflammation of the peritoneum and is typically caused by an infection. Typically, the subject is suffering from kidney disease. In some embodiments, the kidney disease is chronic and/or severe.

Thus, the disclosure is particularly concerned with the detection of infections, a common problem with peritoneal dialysis. Subjects found to be suffering from peritonitis may need to be treated, for example with an appropriate antibiotic.

Accordingly, in a further aspect, the disclosure provides a method of selecting a subject for treatment with an antibiotic comprising determining the level of at least one marker selected from MMP8, HNE, MMP2, MMP9, TIMP1, TIMP2, NGAL, A1AT, desmosine, fibrinogen, IL-6, IL-8, caprotectin, fMLP, IL1b, cystatin C, HSA, RBP4, SPD, MPO, sICAM and TNFa in a sample of peritoneal dialysate wherein an increased level of at least one of MMP8, HNE, MMP2, MMP9, TIMP1, TIMP2, NGAL, A1AT, desmosine, IL-6, IL-8, caprotectin, fMLP, IL1b, cystatin C, HSA, RBP4, SPD, MPO, sICAM and TNFa and/or a decreased level of fibrinogen results in selection of the subject for treatment with an antibiotic.

Similarly, the disclosure further provides in a further aspect a method of predicting responsiveness of a subject to treatment with an antibiotic comprising determining the level of at least one marker selected from MMP8, HNE, MMP2, MMP9, TIMP1, TIMP2, NGAL, A1AT, desmosine, fibrinogen, IL-6, IL-8, caprotectin, fMLP, IL1b, cystatin C, HSA, RBP4, SPD, MPO, sICAM and TNFa in a sample of peritoneal dialysate wherein an increased level of at least one of MMP8, HNE, MMP2, MMP9, TIMP1, TIMP2, NGAL, A1AT, desmosine, IL-6, IL-8, caprotectin, fMLP, IL1b, cystatin C, HSA, RBP4, SPD, MPO, sICAM and TNFa and/or a decreased level of fibrinogen predicts responsiveness of the subject to treatment with an antibiotic.

Any suitable antibiotic may be employed to treat peritonitis as discussed for example in Warady et al (2012) Peritoneal Dialysis International, Vol. 32, pp. S32-S86 doi: 10.3747/pdi.2011.00091 (incorporated herein by reference). Thus, in some embodiments of the disclosure, the antibiotic is selected from an aminoglycoside, a cephalosporin, a glycopeptide, a penicillin, a quinolone, aztreonam, clindamycin, imipenem-cilastin, linezolid, metronidazole, rifampin and an antifungal. Combinations are also envisaged within the scope of the disclosure.

In certain embodiments, the aminoglycoside is selected from gentamicin, netilmycin, tobramycin and amikacin. In some embodiments, the cephalosporin is selected from cefazolin, cefepimine, cefotaxime and ceftazidimine. In some embodiments, the glycopeptide is selected from vancomycin and teicoplanin. In some embodiments, the antifungal is selected from fluconazole and caspofungin.

Any suitable route of administration may be employed. In some embodiments, the antibiotic is to be administered intraperitoneally, orally or intravenously. Intraperitoneal administration may be most convenient for PD patients. In some embodiments, the antibiotic may be administered with the dialysis fluid. However, some antibiotics such as aminoglycosides and penicillins should not be mixed in dialysis fluid because of the potential for inactivation.

Suitable dosing regimens can be readily derived by one of skill in the art taking into account dosing instructions and subject characteristics. Some dosing recommendations useful in the present disclosure are shown in Table 1 below, derived from Warady et al:

Antibiotic Dosing Recommendations^{a} for the Treatment of Peritonitis

| | Therapy type | | |
|---|---|---|---|
| | Continuous^{b} | | |
| Antibiotic type | Loading dose | Maintenance dose | Intermittent^{b} |
| Aminoglycosides (IP)^{c} | | | |
| Gentamicin | 8 mg/L | 4 mg/L | |
| Netilmycin | 8 mg/L | 4 mg/L | Anuric: 0.6 mg/kg |
| Tobramycin | 8 mg/L | 4 mg/L | Non-anuric: 0.75 mg/kg |
| Amikacin | 25 mg/L | 12 mg/L | |
| Cephalosporins (IP) | | | |
| Cefazolin | 500 mg/L | 125 mg/L | 20 mg/kg |
| Cefepime | 500 mg/L | 125 mg/L | 15 mg/kg |
| Cefotaxime | 500 mg/L | 250 mg/L | 30 mg/kg |
| Ceftazidime | 500 mg/L | 125 mg/L | 20 mg/kg |
| Glycopeptides (IP)^{d} | | | |
| Vancomycin | 1000 mg/L | 25 mg/L | 30 mg/kg; |
| | | | repeat dosing: |
| | | | 15 mg/kg every 3-5 days |
| Teicoplanin^{e} | 400 mg/L | 20 mg/L | 15 mg/kg every 5-7 days |
| Penicillins (IP)^{c} | | | |
| Ampicillin | — | 125 mg/L | — |
| Quinalones (IP) | | | |
| Ciprofloxacin | 50 mg/L | 25 mg/L | — |
| Others | | | |
| Aztreonam (IP) | 1000 mg/L | 250 mg/L | — |
| Clindamycin (IP) | 300 mg/L | 150 mg/L | — |
| Imipenem-cilastin (IP) | 250 mg/L | 50 mg/L | — |
| Linezolid (PO) | <5 Years: 30 mg/kg daily, divided into 3 doses | | |
| | 5-11 Years: 20 mg/kg daily, divided into 2 doses | | |
| | ≥12 Years: 600 mg/dose, twice daily | | |
| Metronidazole (PO) | 30 mg/kg daily, divided into 3 doses (maximum: 1.2 g daily) | | |
| Rifampin (PO) | 10-20 mg/kg daily, divided into 2 doses (maximum: 600 mg daily) | | |
| Antifungals | | | |
| Fluconazole (IP, IV, or PO) | 6-12 mg/kg every 24-48 h (maximum: 400 mg daily) | | |
| Caspofungin (IV only) | 70 mg/m² on day 1 | 50 mg/m² daily | |
| | (maximum: 70 mg daily) | (maximum: 50 mg daily) | |

IP = intraperitoneaily; IV = intravenously; PO = orally.
^{a}Adapted from Li et al. (7), The Renal Drug Reference Guide (171), and Taketomo et al. (172).
^{b}For continuous therapy, the exchange with the loading dose should dwell for 3-6 hours; all subsequent exchanges during the
treatment course should contain the maintenance dose. For intermittent therapy, the dose should be applied once daily in the
long-dwell, unless otherwise specified.
^{c}Aminoglycosides and penicillins should not be mixed in dialysis fluid because of the potential for inactivation.
^{d}In patients with residual renal function, glycopeptide elimination may be accelerated. If intermittent therapy is used in such a
setting, the second dose should be time-based on a blood level obtained 2-4 days after the intital dose, Re-dosing should occur
when the blood level is <15 mg/L for vancomycin, or <8 mg/L for teicoplanin. Intermittent therapy is not recommended for
patients with residual renal function unless serum levels of the drug can be monitored in a timely manner.
^{e}Teicoplanin is not currently available in the United States.

While each of the markers described herein have been shown to give useful information in relation to peritonitis detection, certain markers give highly significant results (P value <0.0001). Thus, in some embodiments according to all aspects of the disclosure the at least one marker is selected from MMP8, HNE, MMP2, MMP9, IL-6, caprotectin and MPO. In further embodiments, the at least one marker is selected from MMP8, HNE, MMP2, IL-6, caprotectin and MPO. As described herein, the most specific and sensitive marker determined by the inventors is MMP8. Accordingly, in some embodiments, the at least one marker is MMP8.

According to all aspects of the invention, at least two markers are determined. The two markers may be MMP-8 and IL-6 on one embodiment. In another embodiment, one of the at least two markers is MMP-8. In another embodiment, one of the at least two markers is IL-6.

Increased and decreased levels of the markers are specified in relation to the corresponding peritoneal dialysate from a subject that is not suffering from peritonitis. As is shown herein, significantly increased (or decreased in the case of fibrinogen) levels of the markers can be used to sensitively and specifically identify infection at an early stage. This permits early and effective intervention and reduces unnecessary use of antibiotics where no infection is present. Cut off values for increased markers may be at the level of at least 0.1, 0.2, 0.3, 0.4, 0.5, 0.6, 0.7, 0.8, 0.9, 1, 2, 3, 4, 5, 6, 7, 8, 9, 10, 15, 20, 25, 50 or 100 ng/ml or more depending upon the marker concerned. The skilled person would be well able to determine an appropriate cut-off value given a known set of samples containing infections and control samples. Cut-off values may be adjusted to maximise sensitivity and/or specificity as required. For example, decision tree analysis may be used to determine a suitable cut-off. Background levels of some markers may be sufficiently low that any visible line on the test strip is indicative of peritonitis in some embodiments. Thus, in some embodiments, simple detection of the marker indicates peritonitis (and this is included within the definition of “increased” because the levels are higher than control). Alternatively, the various components of the test line may be included at concentrations such that threshold levels of the marker must be exceeded in order to give a visible test line. In specific embodiments, the threshold for MMP8 is around 0.4, such as 0.418 ng/ml.

The level of the markers may be determined at the level of protein or RNA (specifically mRNA). “Level” is also defined to include determining enzymatic activity of the relevant marker, as appropriate. This in some embodiments, the level of at least one marker is determined by an enzymatic activity assay. In some embodiments, zymography is utilised. This technique measures a number of hydrolytic enzymes, but in some embodiments measures MMP2 and/or MMP9 activity. In some embodiments a MMP substrate assay is utilised. Examples of suitable assays can be found in WO2009/024805, WO2009/063208, WO2007/128980, WO2007/096637, WO2013/156794, WO2013/156795 and WO2015/059487 each of which is incorporated herein by reference.

In some embodiments, the level of at least one marker is determined by an immunoassay. Many suitable immunoassay formats are known in the art, including ELISAs which may be sandwich ELISAs and/or competitive ELISAs.

In specific embodiments, the method is performed in a lateral flow assay format. Generally, therefore, the disclosure relies upon some form of solid support. The solid support may define a liquid flow path for the sample. In specific embodiments, the solid support comprises a chromatographic medium or a capillary flow device. The disclosure may be provided in a test strip format in some embodiments. As discussed herein, the lateral flow test strip incorporates a sample receiving zone (i.e. a region of the strip, towards the upstream end to which the sample is applied). The sample may be directly applied to the sample receiving zone. Alternatively, the sample may be transferred from the fluid sampling chamber of the device to the lateral flow test strip, for example via a wick element.

Downstream of the sample receiving zone is a test line. The test line shows a visible line in the presence of (increased levels of) the at least one marker in the sample. Threshold levels of the marker or markers corresponding to a visible test line may be readily determined by one skilled in the art as discussed above.

Multiple markers may be detected in a single test strip according to the invention. This may involve the use of multiple test lines as defined herein, one for each marker. Thus, the test strip may include a test line for at least two of the markers described herein. One of the markers may be MMP-8. One of the markers may be IL-6. The test strip may include a test line for MMP-8 and IL-6 in preferred embodiments.

This test line comprises immobilized capture molecules. The capture molecules may be pre-immobilized at the test line or may become immobilized as liquid flows through the test strip. In the latter case, immobilization typically involves a specific binding interaction for example between a biotin molecule on the capture molecule and an avidin/streptavidin molecule immobilised in the test line. The capture molecules specifically bind to the marker or markers of interest where protein levels are to be determined. Where enzymatic activity is to be measured, the capture molecules may bind to a substrate or processed substrate molecule. Typically the capture molecules are antibodies or fragments or derivatives thereof.

The captured markers are then detected via a second specific binding interaction with a reporter molecule. The reporter molecule is also typically an antibody or fragment or derivative thereof. The reporter molecule is typically contained in the test strip upstream of the test sample. Upon hydration by the sample, the reporter molecule is carried along the test strip and interacts with the marker if present in the sample. It then becomes immobilised at the test line via the interaction between the capture molecule and the marker. Thus, typically, the immunoassay is a sandwich immunoassay. The reporter molecule may be labelled, either directly or indirectly. Suitable labels include gold particles and fluorophores. Indirect labels may be attached to a further binding molecule.

The test strip may also contain a control line downstream of the test line. Typically, this control line is used to confirm that the test has worked properly (i.e. that the sample has run through the entire test strip, past the test line). The control line may contain immobilized capture molecules in a similar fashion to those used at the test line (i.e. either pre-immobilized or which become immobilised during liquid flow through the device). The control line capture molecules may specifically bind to the reporter molecules. Thus, the reporter molecules that are not captured at the test line flow through and are captured at the test line. Alternatively, a separate control molecule may be included in the test strip. Upon hydration by the sample, the separate control molecule is carried along the test strip and specifically binds to the control line capture molecules. It then becomes immobilised at the control as a consequence. Again, the control molecules may be directly or indirectly labelled.

The test strip may also contain a sump downstream of the control line to absorb excess sample. The methods of the disclosure may be performed using large volumes of sample, because the peritoneal dialysate is of relatively large volume (of the order at least 100, 200, 300, 500, 1000, 2500 ml or more).

Corresponding methods of treatment are envisaged in which the subject has been identified for treatment using the methods and apparatus (and kits) described herein. As discussed above, typically the subject is suffering from kidney disease. The kidney disease may be chronic and/or severe.

Thus, in a further aspect the disclosure provides a method of treating peritonitis comprising administering an antibiotic to the subject suffering from peritonitis, wherein the subject has been selected for treatment by performing a method as described herein.

The disclosure also provides a method of treating peritonitis comprising administering an antibiotic to the subject suffering from peritonitis, wherein the subject displays, in a sample of peritoneal dialysate, an increased level of at least one of MMP8, HNE, MMP2, MMP9, TIMP1, TIMP2, NGAL, A1AT, desmosine, IL-6, IL-8, caprotectin, fMLP, IL1b, cystatin C, HSA, RBP4, SPD, MPO, sICAM and TNFa and/or a decreased level of fibrinogen. In specific embodiments, the subject displays an increased level of at least one of MMP8, HNE, MMP2, MMP9, IL-6, caprotectin and MPO. In further embodiments, the subject displays an increased level of least one of MMP8, HNE, MMP2, IL-6, caprotectin and MPO. In specific embodiments, the subject displays an increased level of MMP8.

These methods may also be specified as medical uses of the antibiotics. Thus, the disclosure provides an antibiotic for use in a method of treating peritonitis, wherein the subject has been selected for treatment by performing a method as described herein.

The disclosure further provides an antibiotic for use in a method of treating peritonitis, wherein the subject displays, in a sample of peritoneal dialysate, an increased level of at least one of MMP8, HNE, MMP2, MMP9, TIMP1, TIMP2, NGAL, A1AT, desmosine, IL-6, IL-8, caprotectin, fMLP, IL1 b, cystatin C, HSA, RBP4, SPD, MPO, sICAM and TNFa and/or a decreased level of fibrinogen. In specific embodiments, the subject displays an increased level of at least one of MMP8, HNE, MMP2, MMP9, IL-6, caprotectin and MPO. In further embodiments, the subject displays an increased level of least one of MMP8, HNE, MMP2, IL-6, caprotectin and MPO. In specific embodiments, the subject displays an increased level of MMP8.

Suitable antibiotics are specified herein together with appropriate modes of administration and dosages. That discussion applies mutatis mutandis to these aspects of the disclosure. Thus, in some embodiments, the antibiotic is selected from an aminoglycoside, a cephalosporin, a glycopeptide, a penicillin, a quinolone, aztreonam, clindamycin, imipenem-cilastin, linezolid, metronidazole, rifampin and an antifungal. In some embodiments, the antibiotic is administered intraperitoneally, orally or intravenously (preferably intraperitoneally). In some embodiments, the antibiotic is administered during peritoneal dialysis. In certain embodiments, the antibiotic is administered with the dialysis fluid utilised in the peritoneal dialysis. In other embodiments, the antibiotic, optionally an aminoglycoside or penicllin, is administered separately from the dialysis fluid utilised in the peritoneal dialysis.

The methods are intended to be integrated with the diagnostic test apparatus and corresponding methods and kit of parts. In particular, the lateral flow test strip of the diagnostic test apparatus, kit of parts and methods may comprise the test strips hereinbefore described. Thus the discussion of those aspects represent preferred embodiments.

The invention may also be defined by reference to the following numbered clauses:
1. A diagnostic test apparatus comprising:
   an elongate housing defining a test strip holder containing a lateral flow test strip;
   a fluid sampling chamber provided with an opening connecting the fluid sampling chamber with the test strip holder;
   a viewing window in the elongate housing allowing reading of one or more portions of the lateral flow test strip; and
   a connector configured to couple the diagnostic test apparatus to a bulk source of fluid.
2. A diagnostic test apparatus as described in clause 1, wherein the connector is configured to establish fluid communication between the bulk source of fluid and the fluid sampling chamber.
3. A diagnostic test apparatus as described in clause 1 or clause 2, wherein the connector comprises an interference fit connector, an adhesive connector, or a clip connector.
4. A diagnostic test apparatus as described in any preceding clause, wherein the connector comprises a tubular element.
5. A diagnostic test apparatus as described in clause 4, wherein the tubular element contains the fluid sampling chamber.
6. A diagnostic test apparatus as described in clause 4 or clause 5, wherein the tubular element comprises a first end and a second end and the fluid sampling chamber is interposed between the first end and the second end.
7. A diagnostic test apparatus as described in clause 6, wherein both the first end and the second end are open to thereby enable a flowing stream of fluid to pass through the fluid sampling chamber during testing.
8. A diagnostic test apparatus as described in clause 6, wherein the first end is open and the second end is closed to thereby enable a volume of fluid to pass into, and be retained within, the fluid sampling chamber during testing.
9. A diagnostic test apparatus as described in any of clauses 4 to 8, wherein the tubular element is configured for push-fit coupling to a tube of the bulk source of fluid.
10. A diagnostic test apparatus as described in clause 9, wherein the tubular element comprises an O-ring seal for engaging the tube of the bulk source of fluid.
11. A diagnostic test apparatus as described in any of clauses 4 to 10, wherein the connector comprises a clip for clipping to a tube of the bulk source of fluid.
12. A diagnostic test apparatus as described in any of clauses 4 to 11, wherein the tubular element comprises an adhesive element for adhering to a surface of a container of the bulk source of fluid.
13. A diagnostic test apparatus as described in clause 12, wherein the adhesive element comprises an adhesive pad provided on an end of the tubular element.
14. A diagnostic test apparatus as described in any preceding clause, wherein the connector is integral with the elongate housing.
15. A diagnostic test apparatus as described in any preceding clause, further comprising a puncturing element for opening fluid communication between the bulk source of fluid and the fluid sampling chamber.
16. A diagnostic test apparatus as described in clause 15, wherein the puncturing element is contained within the fluid sampling chamber.
17. A diagnostic test apparatus as described in clause 15 or clause 16, wherein the puncturing element punctures an element of the bulk source of fluid during coupling of the connector to the bulk source of fluid.
18. A diagnostic test apparatus as described in clause 15 or clause 16, wherein the puncturing element punctures an element of the bulk source of fluid after coupling of the connector to the bulk source of fluid.
19. A diagnostic test apparatus as described in any of clauses 15 to 18, wherein the puncturing element comprises a piercing element.
20. A diagnostic test apparatus as described in clause 19, wherein the piercing element is statically-mounted within the connector.
21. A diagnostic test apparatus as described in clause 19, wherein the piercing element is movably-mounted within the connector to be movable from a retracted position to an extended position.
22. A diagnostic test apparatus as described in clause 21, wherein the piercing element is manually movable into the extended position by operation of a twistable and/or pushable plunger.
23. A diagnostic test apparatus as described in any of clauses 15 to 22, wherein the puncturing element comprises a pin, tube or blade.
24. A diagnostic test apparatus as described in any preceding clause, further comprising a wick element for wicking fluid from the fluid sampling chamber into the test strip holder.
25. A diagnostic test apparatus as described in clause 24, wherein the wick element projects through the opening into the fluid sampling chamber.
26. A diagnostic test apparatus as described in clause 24 or clause 25, wherein the wick element comprises an end portion of the lateral flow test strip.
27. A diagnostic test apparatus as described in clause 24 or clause 25, wherein the wick element comprises a separate wick element that is in fluid contact with the lateral flow test strip.
28. A diagnostic test apparatus as described in any of clauses 24 to 27, wherein the wick element comprises an elongate wick.
29. A diagnostic test apparatus as described in any of clauses 24 to 27, wherein the wick element comprises a tubular or part-tubular element.
30. A diagnostic test apparatus as described in any of clauses 24 to 29, wherein the wick comprises a foam element.
31. A diagnostic test apparatus as described in any preceding clause, wherein the fluid sampling chamber is located at a first end of the elongate housing.
32. A diagnostic test apparatus as described in any preceding clause, wherein the fluid sampling chamber is formed integrally with the elongate housing.
33. A kit of parts comprising:
   a diagnostic test apparatus as described in any preceding clause; and
   a bulk source of fluid.
34. The kit of parts of clause 33, wherein the bulk source of fluid comprises a container holding the fluid and the connector of the diagnostic test apparatus is configured to be coupled to the container.
35. The kit of parts of clause 34, wherein the container is closed prior to coupling of the diagnostic test apparatus.
36. The kit of parts of clause 34 or clause 35, wherein the container comprises a storage bag and one or more tubes extending from the storage bag.
37. The kit of parts of clause 36, wherein the connector of the diagnostic test apparatus is configured to be coupled to the storage bag.
38. The kit of parts of any of clauses 34 to 37, wherein the connector of the diagnostic test apparatus is configured to be coupled to the one or more tubes.
39. The kit of parts of any of clauses 34 to 38, wherein the container holds greater than 100 ml, optionally greater than 250 ml, optionally greater than 1000 ml of fluid.
40. The kit of parts of any of clauses 34 to 39, wherein the container is a dialysate bag, optionally a peritoneal dialysate bag.
41. A method of carrying out a diagnostic test on a bulk source of fluid using a diagnostic test apparatus of the type comprising:
   an elongate housing defining a test strip holder containing a lateral flow test strip, a fluid sampling chamber provided with an opening connecting the fluid sampling chamber with the test strip holder, and a viewing window in the elongate housing allowing reading of one or more portions of the lateral flow test strip;
   the method comprising the steps of:
   coupling the diagnostic test apparatus to the bulk source of fluid by use of a connector;
   during or after coupling of the connector, establishing a fluid communication between the bulk source of fluid and the fluid sampling chamber of the diagnostic test apparatus to convey fluid from the bulk source of fluid into the fluid sampling chamber;
   causing fluid to pass from the fluid sampling chamber through the opening to wet the lateral flow test strip.
42. The method of clause 41, wherein the fluid is wicked from the fluid sampling chamber through the opening.
43. The method of clause 41 of clause 42, wherein the connector is interference fit, adhered, or clipped to the bulk source of fluid.
44. The method of any of clauses 41 to 43, wherein fluid is conveyed from the bulk source of fluid through the fluid sampling chamber as a flowing stream of fluid that passes through the fluid sampling chamber during testing.
45. The method of any of clauses 41 to 43, wherein fluid is conveyed from the bulk source of fluid into, and retained within, the fluid sampling chamber during testing.
46. The method of any of clauses 41 to 46, wherein the fluid communication between the bulk source of fluid and the fluid sampling chamber of the diagnostic test apparatus is established by puncturing an element of the bulk source of fluid.
47. The method of clause 46, wherein the element of the bulk source of fluid is punctured during coupling of the connector to the bulk source of fluid.
48. The method of clause 46, wherein the element of the bulk source of fluid is punctured after coupling of the connector to the bulk source of fluid.
49. The method of any of clauses 46 to 48, wherein the element of the bulk source of fluid is punctured by driving the element against a puncturing element of the connector.
50. The method of any of clauses 46 to 48, wherein the element of the bulk source of fluid is punctured by driving a puncturing element of the connector through the element of the bulk source of fluid.
51. The method of any of clauses 41 to 50, wherein the bulk source of fluid comprises a container holding the fluid and the connector of the diagnostic test apparatus is configured to be coupled to the container.
52. The method of clause 51, wherein the container is closed prior to coupling of the diagnostic test apparatus.
53. The method of clause 51 or clause 52, wherein the container is a dialysate bag, optionally a peritoneal dialysate bag.
54. The method of any of clauses 41 to 50, wherein the bulk source of fluid comprises a continuous flow of fluid along a drain line and the connector of the diagnostic test apparatus is configured to be coupled to the drain line.
59. A diagnostic test apparatus substantially as hereinbefore described with reference to and as shown in the accompanying drawings.
60. A method of detecting peritonitis in a subject comprising determining the level of at least one marker selected from MMP8, HNE, MMP2, MMP9, TIMP1, TIMP2, NGAL, A1AT, desmosine, fibrinogen, IL-6, IL-8, calprotectin, fMLP, MM1b, cystatin C, HSA, RBP4, SPD, MPO, sICAM and TNFa in a sample of peritoneal dialysate wherein an increased level of at least one of MMP8, HNE, MMP2, MMP9, TIMP1, TIMP2, NGAL, A1AT, desmosine, IL-6, IL-8, calprotectin, fMLP, IL1b, cystatin C, HSA, RBP4, SPD, MPO, sICAM and TNFa and/or a decreased level of fibrinogen is indicative of peritonitis.
61. A method of selecting a subject for treatment with an antibiotic comprising determining the level of at least one marker selected from MMP8, HNE, MMP2, MMP9, TIMP1, TIMP2, NGAL, A1AT, desmosine, fibrinogen, IL-6, IL-8, calprotectin, fMLP, IL1b, cystatin C, HSA, RBP4, SPD, MPO, sICAM and TNFa in a sample of peritoneal dialysate wherein an increased level of at least one of MMP8, HNE, MMP2, MMP9, TIMP1, TIMP2, NGAL, A1AT, desmosine, IL-6, IL-8, calprotectin, fMLP, IL1b, cystatin C, HSA, RBP4, SPD, MPO, sICAM and TNFa and/or a decreased level of fibrinogen results in selection of the subject for treatment with an antibiotic.
62. A method of predicting responsiveness of a subject to treatment with an antibiotic comprising determining the level of at least one marker selected from MMP8, HNE, MMP2, MMP9, TIMP1, TIMP2, NGAL, A1AT, desmosine, fibrinogen, IL-6, IL-8, calprotectin, fMLP, IL1b, cystatin C, HSA, RBP4, SPD, MPO, sICAM and TNFa in a sample of peritoneal dialysate wherein an increased level of at least one of MMP8, HNE, MMP2, MMP9, TIMP1, TIMP2, NGAL, A1AT, desmosine, IL-6, IL-8, calprotectin, fMLP, IL1b, cystatin C, HSA, RBP4, SPD, MPO, sICAM and TNFa and/or a decreased level of fibrinogen predicts responsiveness of the subject to treatment with an antibiotic.
63. The method of clause 61 or clause 62, wherein the antibiotic is selected from an aminoglycoside, a cephalosporin, a glycopeptide, a penicillin, a quinolone, aztreonam, clindamycin, imipenem-cilastin, linezolid, metronidazole, rifampin and an antifungal.
64. The method of any of clauses 61 to 63, wherein the antibiotic is to be administered intraperitoneally, orally or intravenously (preferably intraperitoneally).
65. The method of any of clauses 60 to 64, wherein the at least one marker is selected from MMP8, HNE, MMP2, MMP9, IL-6, calprotectin and MPO.
66. The method of any of clauses 60 to 64, wherein the at least one marker is selected from MMP8, HNE, MMP2, IL-6, calprotectin and MPO.
67. The method of any of clauses 60 to 64, wherein the at least one marker is MMP8.
68. The method of any of clauses 60 to 67, wherein the level of at least one marker is determined by an immunoassay.
69. The method of any of clauses 60 to 68, wherein the level of at least one marker is determined by an enzymatic activity assay.
70. The method of any of clauses 60 to 69, and which is performed by a lateral flow assay.
71. The method of any of clauses 60 to 70, wherein the subject is suffering from kidney disease.
72. The method of any of clauses 60 to 71, wherein the kidney disease is chronic and/or severe.
73. The method of any of clauses 60 to 72, which is performed according to the method of any one of clauses 41 to 54.
74. The method of any of clauses 60 to 72, which is performed using the diagnostic test apparatus of any one of clauses 1 to 32.
75. The method of any of clauses 60 to 72, which is performed using the kit of parts of any one of clauses 33 to 40.
76. A method of treating peritonitis comprising administering an antibiotic to the subject suffering from peritonitis, wherein the subject has been selected for treatment by performing the method of any one of clauses 60 to 75.
77. A method of treating peritonitis comprising administering an antibiotic to the subject suffering from peritonitis, wherein the subject displays, in a sample of peritoneal dialysate, an increased level of at least one of MMP8, HNE, MMP2, MMP9, TIMP1, TIMP2, NGAL, A1AT, desmosine, IL-6, IL-8, calprotectin, fMLP, IL1b, cystatin C, HSA, RBP4, SPD, MPO, sICAM and TNFa and/or a decreased level of fibrinogen.
78. The method of clause 77, wherein the subject displays an increased level of at least one of MMP8, HNE, MMP2, MMP9, IL-6, calprotectin and MPO.
79. The method of clause 77, wherein the subject displays an increased level of least one of MMP8, HNE, MMP2, IL-6, calprotectin and MPO.
80. The method of clause 77, wherein the subject displays an increased level of MMP8.
81. The method of any of clauses 76 to 80, wherein the antibiotic is selected from an aminoglycoside, a cephalosporin, a glycopeptide, a penicillin, a quinolone, aztreonam, clindamycin, imipenem-cilastin, linezolid, metronidazole, rifampin and an antifungal.
82. The method of any of clauses 76 to 81, wherein the antibiotic is administered intraperitoneally, orally or intravenously (preferably intraperitoneally).
83. The method of any of clauses 76 to 82, wherein the antibiotic is administered during peritoneal dialysis.
84. The method of any of clauses 76 to 83, wherein the antibiotic is administered with the dialysis fluid utilised in the peritoneal dialysis.
85. The method of any of clauses 76 to 83, wherein the antibiotic, optionally an aminoglycoside or penicllin, is administered separately from the dialysis fluid utilised in the peritoneal dialysis.
86. The method of any of clauses 76 to 85, wherein the subject is suffering from kidney disease.
87. The method of clause 86, wherein the kidney disease is chronic and/or severe.
88. An antibiotic for use in a method of treating peritonitis, wherein the subject has been selected for treatment by performing the method of any one of clauses 60 to 75.
89. An antibiotic for use in a method of treating peritonitis, wherein the subject displays, in a sample of peritoneal dialysate, an increased level of at least one of MMP8, HNE, MMP2, MMP9, TIMP1, TIMP2, NGAL, A1AT, desmosine, IL-6, IL-8, calprotectin, fMLP, IL1b, cystatin C, HSA, RBP4, SPD, MPO, sICAM and TNFa and/or a decreased level of fibrinogen.
90. The antibiotic for use of clause 89, wherein the subject displays an increased level of at least one of MMP8, HNE, MMP2, MMP9, IL-6, calprotectin and MPO.
91. The antibiotic for use of clause 89, wherein the subject displays an increased level of least one of MMP8, HNE, MMP2, IL-6, calprotectin and MPO.
92. The antibiotic for use of clause 89, wherein the subject displays an increased level of MMP8.
93. The antibiotic for use of clause 89, wherein the antibiotic is selected from an aminoglycoside, a cephalosporin, a glycopeptide, a penicillin, a quinolone, aztreonam, clindamycin, imipenem-cilastin, linezolid, metronidazole, rifampin and an antifungal.
94. The antibiotic for use of any of clauses 89 to 93, wherein the antibiotic is administered intraperitoneally, orally or intravenously (preferably intraperitoneally).
95. The antibiotic for use of any of clauses 89 to 94, wherein the antibiotic is administered during peritoneal dialysis.
96. The antibiotic for use of any of clauses 89 to 95, wherein the antibiotic is administered with the dialysis fluid utilised in the peritoneal dialysis.
97. The antibiotic for use of any of clauses 89 to 95, wherein the antibiotic, optionally an aminoglycoside or penicllin, is administered separately from the dialysis fluid utilised in the peritoneal dialysis.
98. The antibiotic for use of any of clauses 89 to 97, wherein the subject is suffering from kidney disease.
99. The antibiotic for use of clause 98, wherein the kidney disease is chronic and/or severe.

### BRIEF DESCRIPTION OF THE DRAWINGS

The present disclosure will now be described, by way of example only, with reference to the accompanying drawings in which:

FIG. 1: A schematic representation of peritoneal dialysis;

FIGS. 2 to 5: A first embodiment of diagnostic test apparatus according to the present disclosure;

FIGS. 6 to 9: A second embodiment of diagnostic test apparatus according to the present disclosure;

FIGS. 10 to 13: A third embodiment of diagnostic test apparatus according to the present disclosure;

FIGS. 14 to 19: A fourth embodiment of diagnostic test apparatus according to the present disclosure;

FIG. 20A: Scatter graph showing median and interquartile range (IQR) generated from ELISA data comparing peritonitis vs stable individuals (Mann-Whitney test p<0.0001);

FIG. 20B: Receiver Operator Characteristic (ROC) curves for the diagnosis of infection, area under the curve (AUC) values are shown;

FIG. 21A: Scatter graph showing median and IQR generated from LF data comparing peritonitis vs stable individuals (Mann-Whitney test p<0.0001);

FIG. 21B: ROC curve for the diagnosis of infection;

FIG. 22: Decision tree analysis providing initial cut-off for levels of MMP8;

FIG. 23A: Scatter graph showing median and IQR generated from zymography data comparing peritonitis vs stable individuals (Mann-Whitney test p<0.0001);

FIG. 23B: Receiver Operator Characteristic (ROC) curves for the diagnosis of infection, area under the curve (AUC) values are shown;

FIG. 24A: Scatter graph showing median and IQR generated from MMP9 data comparing peritonitis vs stable individuals (Mann-Whitney test p<0.0001);

FIG. 24B: Receiver Operator Characteristic (ROC) curves for the diagnosis of infection, area under the curve (AUC) values are shown;

FIG. 25A: Scatter graph showing median and IQR generated from HNE data comparing peritonitis vs stable individuals (Mann-Whitney test p<0.0001);

FIG. 25B: Receiver Operator Characteristic (ROC) curves for the diagnosis of infection, area under the curve (AUC) values are shown;

FIG. 26A: Scatter graph showing median and IQR generated from IL6 data comparing peritonitis vs stable individuals (Mann-Whitney test p<0.0001);

FIG. 26B: Receiver Operator Characteristic (ROC) curves for the diagnosis of infection, area under the curve (AUC) values are shown;

FIG. 27A: Scatter graph showing median and IQR generated from IL8 data comparing peritonitis vs stable individuals (Mann-Whitney test p<0.0001);

FIG. 27B: Receiver Operator Characteristic (ROC) curves for the diagnosis of infection, area under the curve (AUC) values are shown;

FIG. 28A: Scatter graph showing median and IQR generated from calprotectin data comparing peritonitis vs stable individuals (Mann-Whitney test p<0.0001);

FIG. 28B: Receiver Operator Characteristic (ROC) curves for the diagnosis of infection, area under the curve (AUC) values are shown;

FIG. 29A: Scatter graph showing median and IQR generated from MPO data comparing peritonitis vs stable individuals (Mann-Whitney test p<0.0001); and

FIG. 29B: Receiver Operator Characteristic (ROC) curves for the diagnosis of infection, area under the curve (AUC) values are shown.

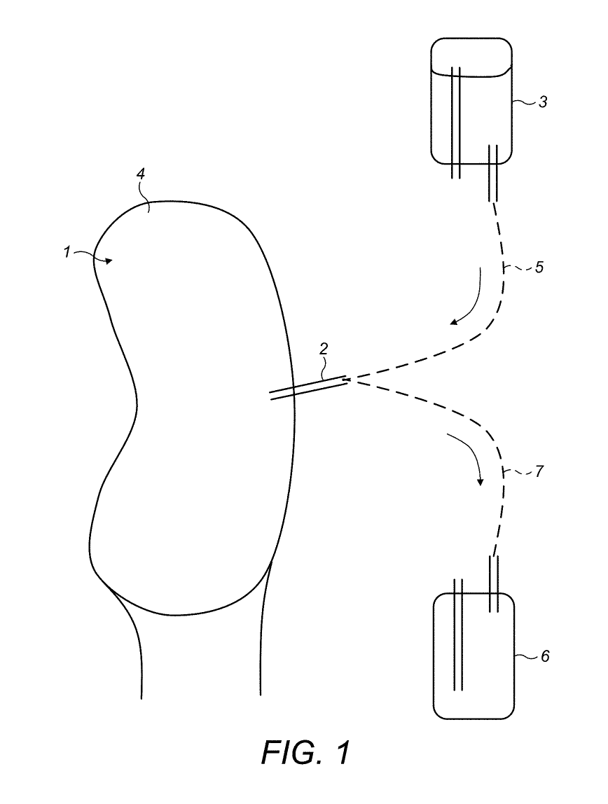

### DETAILED DESCRIPTION

In the following the present disclosure will be described by way of example only for the testing of dialysate fluid for peritonitis infection where the dialysate fluid is contained in or flowing into a dialysate bag. It will be readily appreciated that the diagnostic test apparatus, kit of parts and methods herein may be utilised with other fluids and other containers and for testing for different substances and conditions.

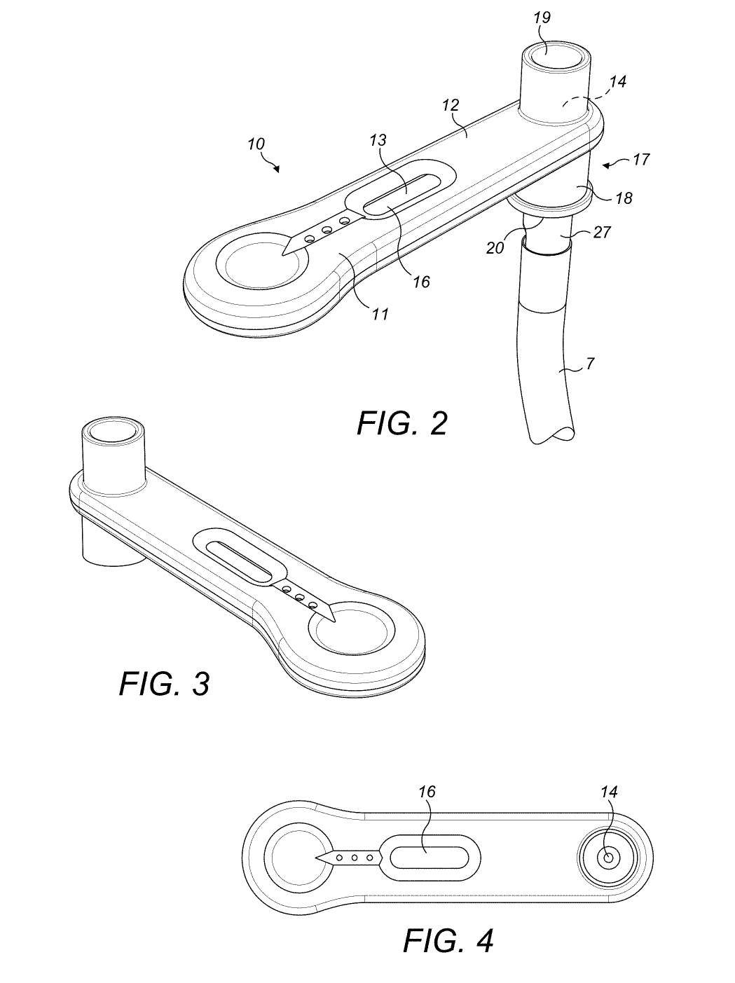

A first embodiment of a diagnostic test apparatus 10 according to the present disclosure is shown in FIGS. 2 to 5 and comprises an elongate housing 11 defining a test strip holder 12 which contains a lateral flow test strip 13. A fluid sampling chamber 14 is provided that connects with the test strip holder 12 via an internal fluid opening. The elongate housing 11 is provided with a viewing window 16 allowing viewing and reading of a portion of the lateral flow test strip 13.

The lateral flow test strip 13 may project into the fluid sampling chamber 14 through the opening. Alternatively, a wick element may be provided for wicking fluid from the fluid sampling chamber through the opening into contact with the lateral flow test strip 13.

A connector 17 is provided for coupling the diagnostic test apparatus 10 to a bulk source of fluid. The connector 17 may be located at one end of the elongate housing 11 and may be formed integrally, or partly integrally therewith.

The connector 17 may comprise a tubular element 18 having an open first end 19 and an open second end 20. The fluid sampling chamber 14 may be located interposed between the first end 19 and the second end 20. As shown, the second end 20 may be coupled to a drain line 7 leading to a dialysate fluid bag. The first end 19 may be coupled to another drain line (not shown) for feeding fluid from a patient's abdomen via a catheter.

The connector 17 may comprise more than one piece. As shown, either or both of the first end 19 and second end 20 may comprise a separate tubular interconnector 27 allowing an interference push-fit coupling of the tubular element 18 with the drain lines. Alternatively, the size and shape of the first end 19 and second end 20 may be configured to directly receive the drain lines as an interference push-fit.

In use, fluid to be tested—for example fluid received from a drain line connected to a catheter—passes through the diagnostic test apparatus 10 as a stream of fluid passing from a first drain line fluidly coupled to the first end 19 of the connector 17 via the fluid sampling chamber 14 and out of the second end 20 of the connector 17 into the fluidly coupled drain line 7. The fluid flowing through the fluid sampling chamber wets the lateral flow test strip 13 either directly or via wetting of the intermediate wick element.

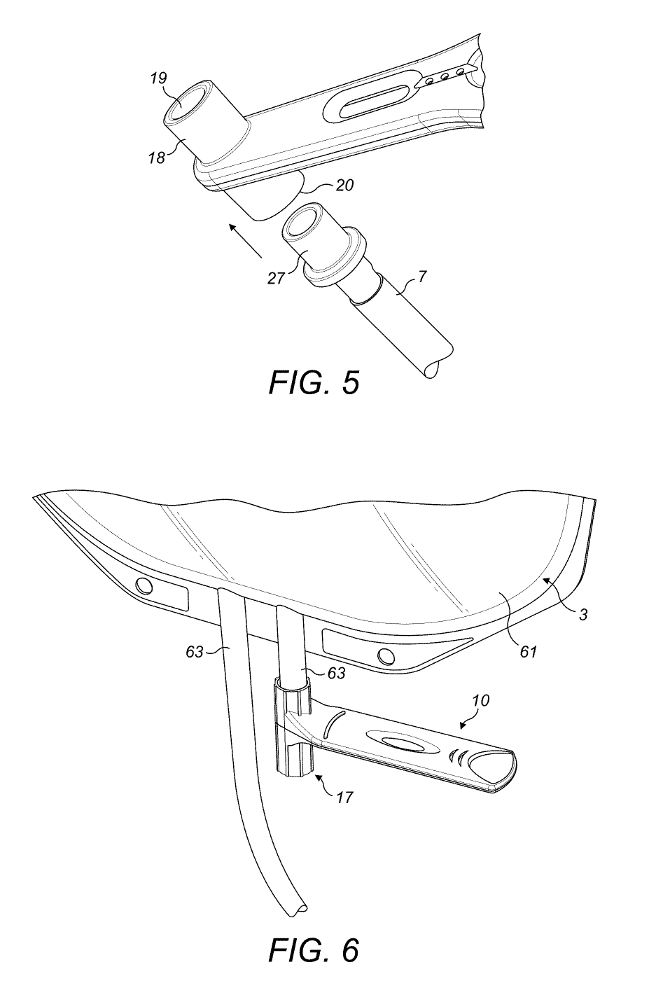

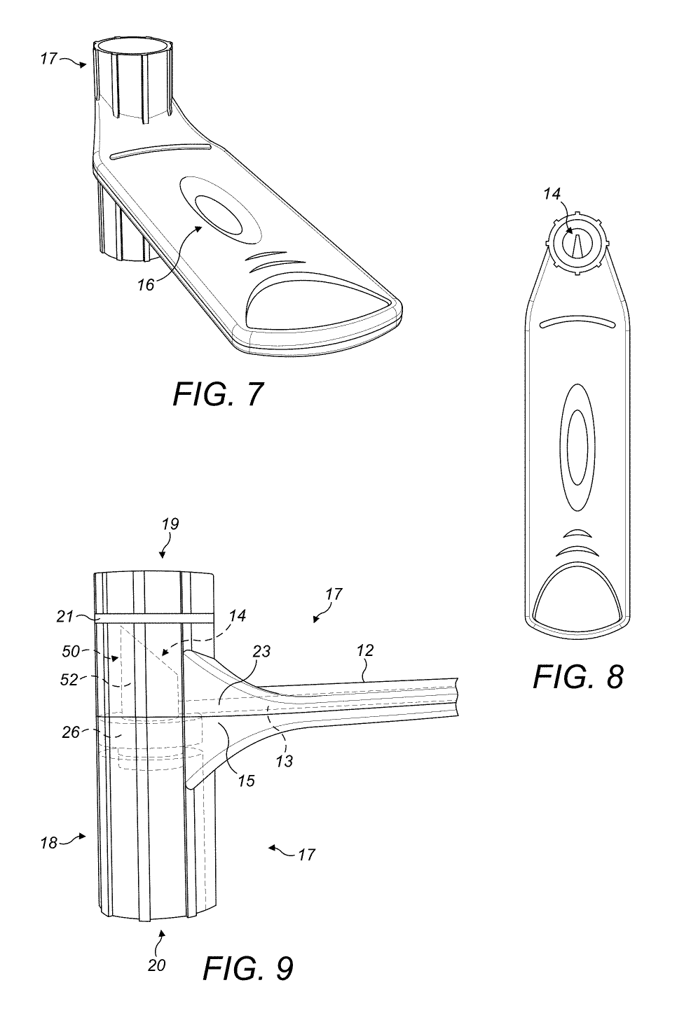

A second embodiment of a diagnostic test apparatus 10 according to the present disclosure is shown in FIGS. 6 to 9. Like reference numerals have been used for like components between embodiments. As with the first embodiment, the diagnostic test apparatus 10 comprises an elongate housing 11 defining a test strip holder 12 which contains the lateral flow test strip 13 and is provided with a viewing window 16 allowing viewing and reading of a portion of the lateral flow test strip 13.

As above, a connector 17 is provided for coupling the diagnostic test apparatus 10 to a bulk source of fluid. In this example the bulk source of fluid is shown as a dialysate fluid bag 3. The dialysate fluid bag 3 may comprise a flexible sac 61 having one or more tubes 63 extending therefrom. The connector 17 may be configured for coupling to an end of one of the tubes 63.

The connector 17 may be located at one end of the elongate housing 11 and may be formed integrally, or partly integrally therewith. The connector 17 may comprise a tubular element 18 as above having an open first end 19. However, in this embodiment the second end 20 of the tubular element 18 is closed. As most clearly seen in FIG. 9, the fluid sampling chamber 14 is interposed between the open first end 19 and the closed second end 20. FIG. 9 also illustrates the opening 15 which may be internal and which connects the fluid sampling chamber 14 with the test strip holder 12. The lateral flow test strip 13 may project into the fluid sampling chamber 14 through the opening 15. Alternatively, a wick element 23 may be provided for wicking fluid from the fluid sampling chamber 14 through the opening 15 into contact with the lateral flow test strip 13.

The connector 17 also contains a puncturing element 50 for establishing fluid communication with the dialysate fluid bag 3. In the example shown the puncturing element 50 comprises a piercing tube 52, which may be hollow and may be statically-mounted within the fluid sampling chamber 14. An O-ring seal 21 may be provided at the open first end 19.

The fluid sampling chamber 14 may contain a foam element 26. The foam element 26 may be a foam ring that surrounds the piercing tube 52. The foam element 26 may assist in transferring fluid from the fluid sampling chamber 14 to the wick element 23. Alternatively, the foam element 26 may act itself as the wick element and be in direct contact with the lateral flow test strip 13.

As shown, the first end 19 may be coupled to an end of a tube 63 of the dialysate fluid bag 3. Typically the tube 63 will be initially closed off to prevent leakage of fluid by means of a bung. In order to couple the connector 17 to the dialysate fluid bag 3, the end of the tube 63 is pushed into the open first 19 of the connector 17 causing puncturing of the bung by the piercing tube 52. In this way fluid communication between the dialysate fluid bag 3 and the fluid sampling chamber 14 is established allowing fluid to flow into the fluid sampling chamber 14 to wet the lateral flow test strip 13 either directly or via the wick element 23 and/or the foam element 26. The closed second end 20 of the tubular element 18 and the engagement of the O-ring seal 21 on the tube 63 prevents leakage of fluid outside the diagnostic test apparatus 10.

The foam element 26 may also absorb and retain a fluid sample within the diagnostic test apparatus 10. This may allow the diagnostic test apparatus 10 to be retained for later testing, re-testing or analysis of the fluid sample at a remote site. Suitable means may be provided, for example a push-fit cap, for closing off the open first end 19 of the tubular element 18 after the initial test to assist in retaining the fluid sample.

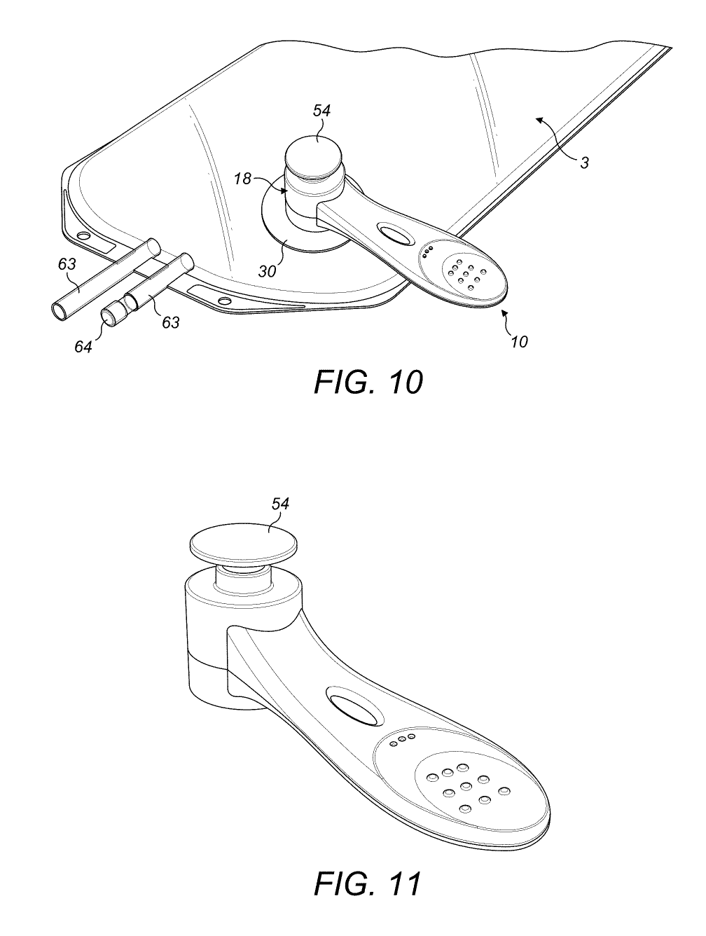

A third embodiment of a diagnostic test apparatus 10 according to the present disclosure is shown in FIGS. 10 to 13. Like reference numerals have been used for like components between embodiments. As with the previous embodiments, the diagnostic test apparatus 10 comprises an elongate housing 11 defining a test strip holder 12 which contains the lateral flow test strip 13 and is provided with a viewing window 16 allowing viewing and reading of a portion of the lateral flow test strip 13.

As above, a connector 17 is provided for coupling the diagnostic test apparatus 10 to a bulk source of fluid. In this example the bulk source of fluid is again shown as a dialysate fluid bag 3 having a flexible sac 61 with one or more tubes 63 extending therefrom. The tubes 63 may be closed off using bungs 64.

In this embodiment the connector 17 may be configured for coupling directly to the flexible sac 61, for example to a surface of the flexible sac 61.

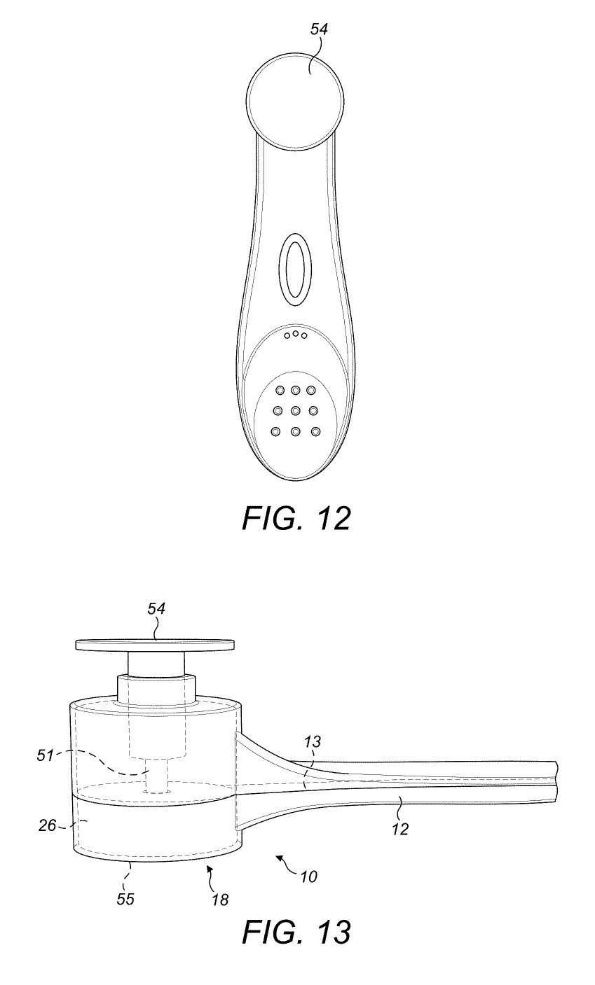

The connector 17 may be located at one end of the elongate housing 11 and may be formed integrally, or partly integrally therewith. The connector 17 may comprise a tubular element 18 defining a fluid sampling chamber 14 therein. The connector 17 also contains a puncturing element 50 for establishing fluid communication with the dialysate fluid bag 3. In the example shown the puncturing element 50 comprises a hollow piercing pin 51 within the fluid sampling chamber 14. The hollow piercing pin 51 may be movably-mounted to be movable between a retracted position, as shown in FIG. 13, wherein the hollow piercing pin 51 is fully within the fluid sampling chamber and an extended position in which the hollow piercing pin 51 is driven outwards through an aperture provided in the otherwise closed second end 20 of the tubular element 18. The first end 19 of the tubular element 18 is provided with a plunger 54 which is operatively connected to the hollow piercing pin 51. The plunger 54 may be operated by pushing or twisting the plunger 54.

As above, the fluid sampling chamber 14 is interposed between the first end 19 and the second end 20 of the tubular element 18. As above, the lateral flow test strip 13 may project into the fluid sampling chamber 14 through the opening 15 or alternatively, a wick element 23 may be provided for wicking fluid from the fluid sampling chamber 14 through the opening 15 into contact with the lateral flow test strip 13.

The fluid sampling chamber 14 may contain a foam element 26. The foam element 26 may be a foam ring that surrounds the hollow piercing pin 51. The foam element 26 may assist in transferring fluid from the fluid sampling chamber 14 to the wick element 23. Alternatively, the foam element 26 may act itself as the wick element and be in direct contact with the lateral flow test strip 13.

As shown in FIG. 10, the second end 20 may be provided with an adhesive pad 30 for adhering the connector 17 to a wall of the flexible sac 61. A further seal element, for example an O-ring seal, may be provided on an underside of the adhesive pad 30.

After coupling of the connector 17 to the flexible sac 61, fluid communication between the dialysate fluid bag 3 and the fluid sampling chamber 14 may be established by operating the plunger 54 to drive the hollow piercing pin 51 into the extended position in which it is driven through and punctures the wall of the flexible sac 61 allowing fluid to flow into the fluid sampling chamber 14 via the foam element 26 and/or the wick element 23.

The foam element 26 may also absorb and retain a fluid sample within the diagnostic test apparatus 10. This may allow the diagnostic test apparatus 10 to be retained for later testing, re-testing or analysis of the fluid sample at a remote site. Suitable means may be provided, for example an adhesive seal, for closing off the aperture in the second end 20 of the tubular element 18 after the initial test to assist in retaining the fluid sample.

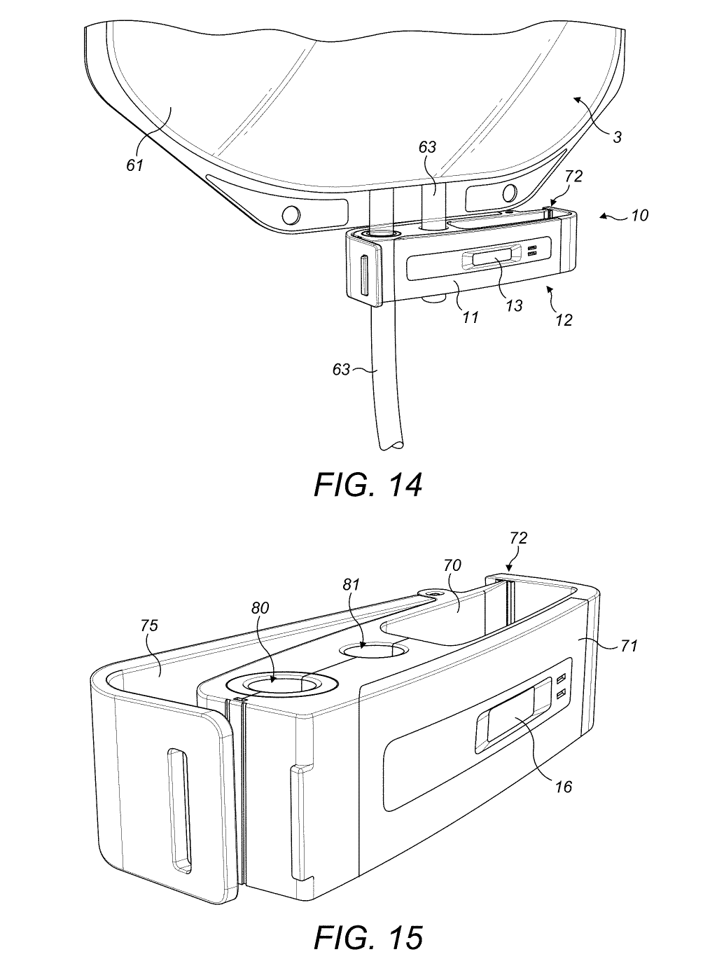

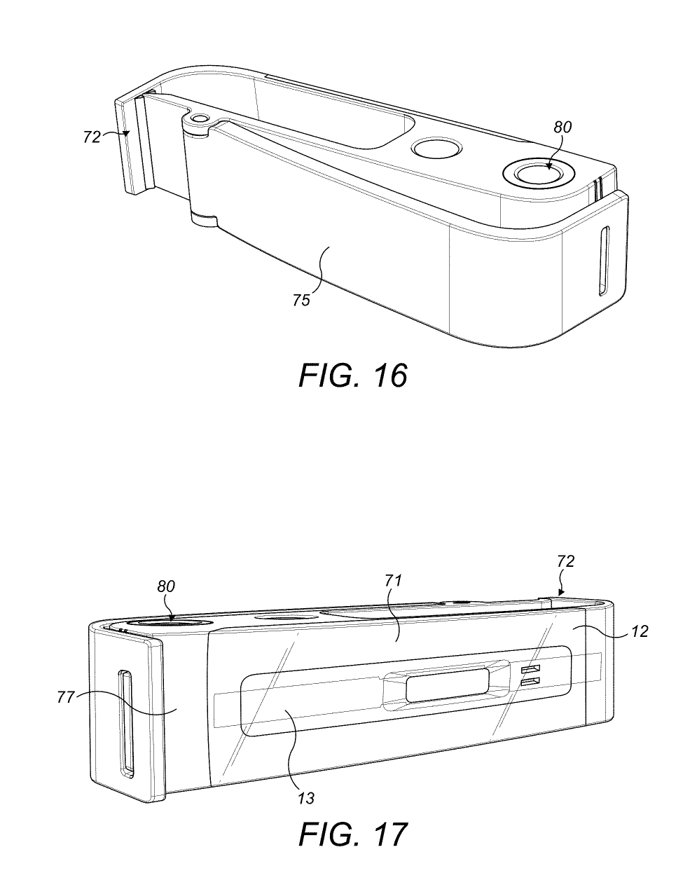

A fourth embodiment of a diagnostic test apparatus 10 according to the present disclosure is shown in FIGS. 14 to 19. Like reference numerals have been used for like components between embodiments. As with the previous embodiments, the diagnostic test apparatus 10 comprises an elongate housing 11 defining a test strip holder 12 which contains the lateral flow test strip 13 and is provided with a viewing window 16 allowing viewing and reading of a portion of the lateral flow test strip 13.

As above, a connector 17 is provided for coupling the diagnostic test apparatus 10 to a bulk source of fluid. In this example the bulk source of fluid is again shown in FIG. 14 as a dialysate fluid bag 3 having a flexible sac 61 with one or more tubes 63 extending therefrom. The tubes 63 may be closed off using bungs 64.

In this embodiment the connector 17 may be configured for coupling directly to one or both tubes 63 with a clipping action.

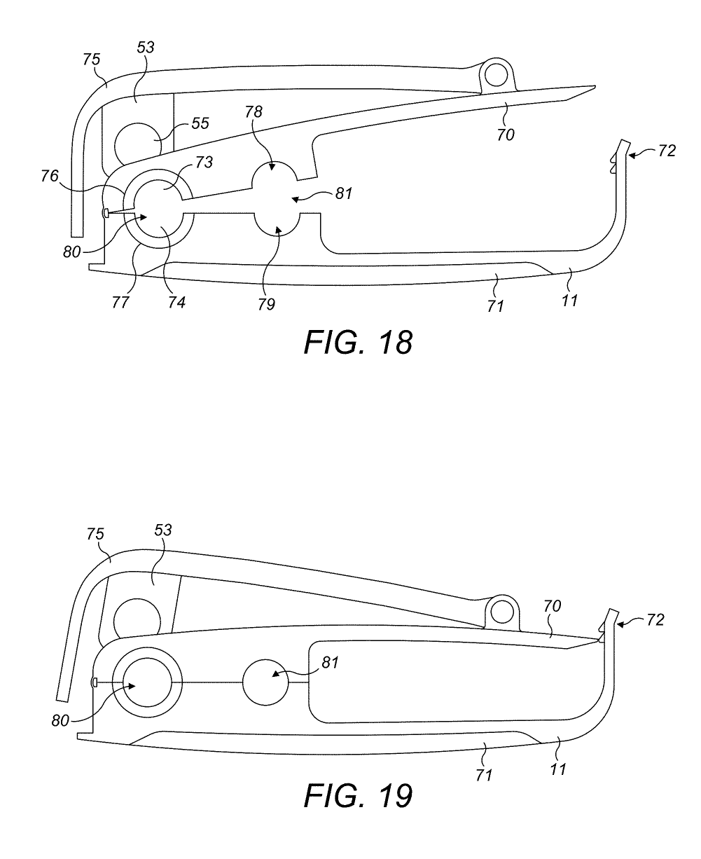

The diagnostic test apparatus 10 may comprise two clamping legs 70, 71 wherein one of the clamping legs may comprise the elongate housing 11. The two clamping legs 70, 71 may be moved between an open configuration as shown in FIG. 18 and a closed configuration as shown in FIG. 19. A retention clip 72 may be provided at the distal ends of the clamping legs 70, 71 which allows the diagnostic test apparatus 10 to be at least temporarily retained in the closed configuration without manual intervention.

The connector 17 may comprise a tubular element 18 formed by the interaction of the two clamping legs 70, 71. As shown in FIGS. 15 and 18, each clamping leg may be provided with a part-tubular recess 73, 74, optionally semi-tubular recesses, that when brought together in the closed configuration define a tubular cavity 80 therebetween. The tubular cavity 80 may be provided with a foam element 26 which may be in the form of two part-tubular foam pieces 76, 77 lining the two part-tubular recesses 73, 74. In the closed configuration the two part-tubular foam pieces 76, 77 may be brought into contact and alignment to provide a tubular foam element that lines the tubular cavity 80.

Optionally, each clamping leg 70, 71 may further be provided with an additional part-tubular recess 78, 79, optionally semi-tubular recesses, that when brought together in the closed configuration define an additional tubular cavity 81 therebetween.

The elongate housing 11 as shown in FIG. 17 comprises a fluid sampling chamber 14 that communicates with or is formed by the part-tubular foam piece 77 in the tubular cavity 80 and also the test strip holder 12 via an opening 15. As above, the lateral flow test strip 13 may extend directly through the opening 15 into contact with the foam element 26 in the form of the tubular foam element or a wick element 23 may be interposed.

The connector 17 also contains a puncturing element 50 for establishing fluid communication with the dialysate fluid bag 3. In the example shown the puncturing element 50 comprises a blade 53 which is movable into the tubular cavity 80 when the clamping legs 70, 71 are in the closed configuration. As shown in FIG. 18, the blade 53 may be mounted on a pivotable third leg 75 which may be pivotally connected to one of the clamping legs 70, 71. The blade 53 may be pivoted into the tubular cavity 80 of the tubular element 18 though an aperture provided in the one of the clamping legs 70, 71.

The diagnostic test apparatus 10 may be coupled to one or two tubes 63 of the dialysate fluid bag 3 as shown in FIG. 14 by clipping the tubes 63 into the tubular cavity 80 and additional tubular cavity 81.

After or during coupling of the connector 17 to the one or more tubes 63, fluid communication between the dialysate fluid bag 3 and the fluid sampling chamber 14 may be established by pivoting the third leg 75 to drive the blade 53 into the tubular cavity 80 wherein it is driven through and punctures the wall of the tube 63 allowing fluid to flow into the tubular cavity 80 where it soaks the tubular foam element. Fluid is then transferred via the tubular foam element onto the lateral flow test strip 13.

The blade 53 may be provided with a central aperture 55 which allows fluid to pass across the plane of the blade 53. This may allow, after puncturing of the tube 63, fluid to be drained from the dialysate fluid bag 3 why carrying out the test. In other words fluid may freely flow through the tubular cavity 80 to drain while wetting the foam element sufficiently to transfer fluid to the lateral flow test strip 13.

The foam element 26 may also absorb and retain a fluid sample within the diagnostic test apparatus 10. This may allow the diagnostic test apparatus 10 to be retained for later testing, re-testing or analysis of the fluid sample at a remote site. Suitable means may be provided, for example adhesive seals or caps, for closing off the tubular cavity 80 after the initial test to assist in retaining the fluid sample.

### Example 1—Pilot Data to Show Selection of Biomarkers

Waste PD fluid was collected from 91 patients with peritonitis and 30 stable patients and stored at −80° C. All samples were analysed for more than 50 potential biomarkers using a variety of reference assays (immunoassays, zymography and protease substrate assays).

Of the potential biomarkers analysed MMP8 was found to be significantly elevated in peritonitis patients compared to stable PD patients.

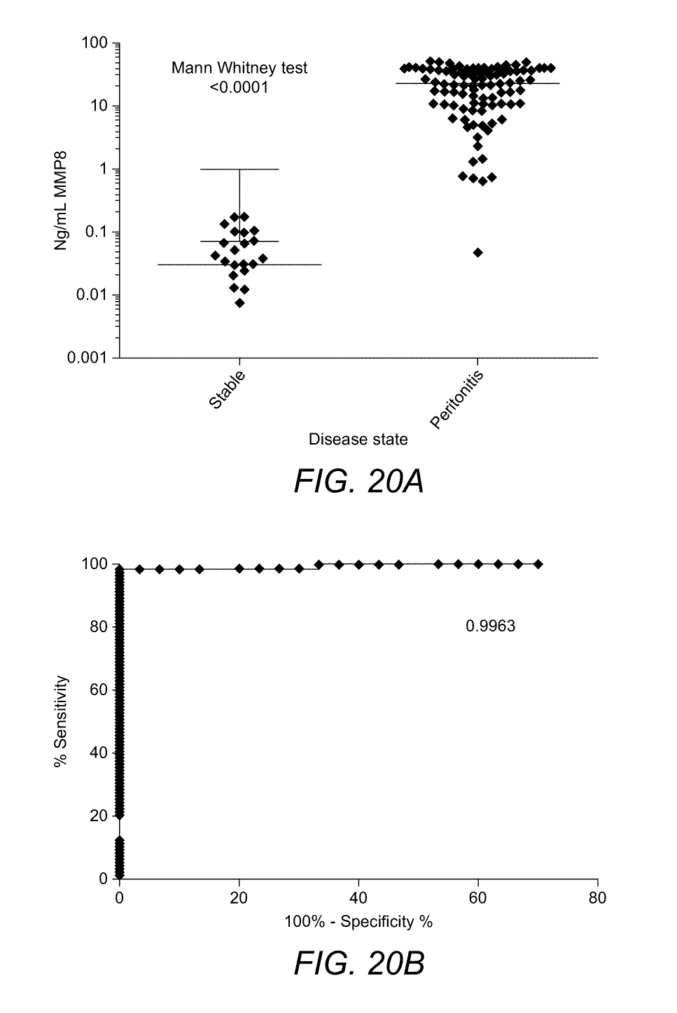

The median level of MMP8 levels in waste PD fluid was 0.032 ng/mL (IQR=0.0 ng/mL-0.076 ng/mL) compared to 23.09 ng/mL (IQR=10.62 ng/mL-37.91 ng/mL) in individuals suffering from peritonitis (FIG. 20A). This marker has excellent diagnostic accuracy with a ROC AUC of 0.9963 (positive predictive value=1.00, negative predictive value=0.97; FIG. 20B and table 2).

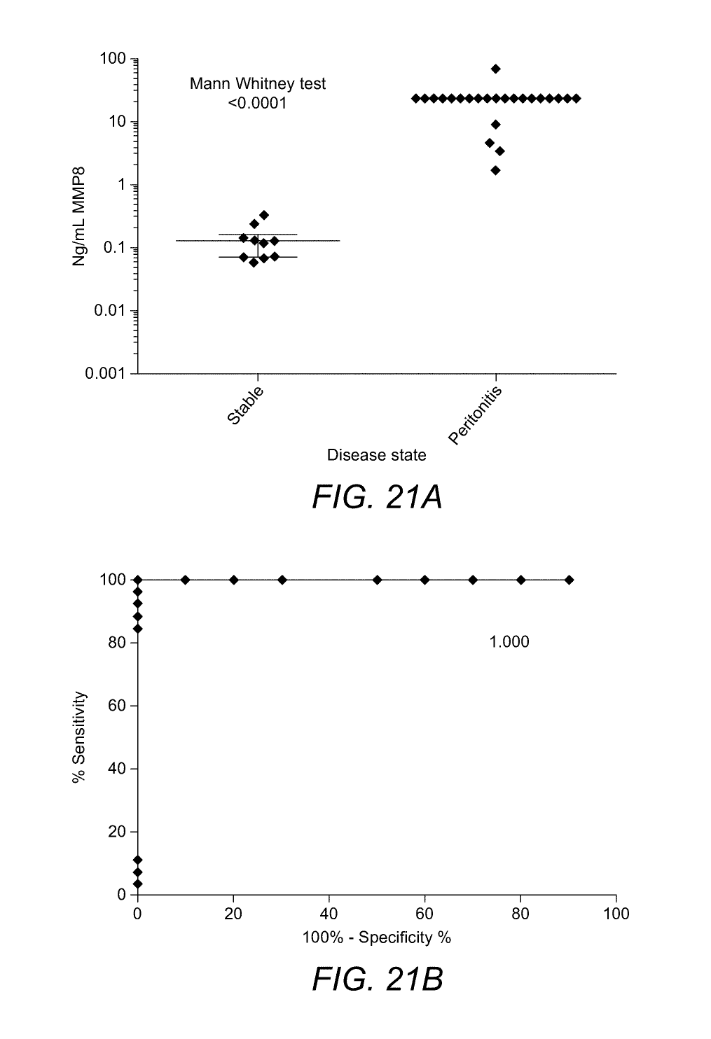

The ability of the assay to perform in a lateral flow format was investigated. For a subset of the samples (peritonitis n=26 and stable n=10) the LF devices produced equivalent results to the ELISA (Spearman's rank=0.808, p<0.001) with an ROC AUC of 1 and a p value of <0.001. Results are shown in FIGS. 21A and 21B.

MMP8 Decision Tree to determine cut-off values:

The preliminary study provided a cut-off value of 0.418 ng/mL using decision tree analysis (SPSS).

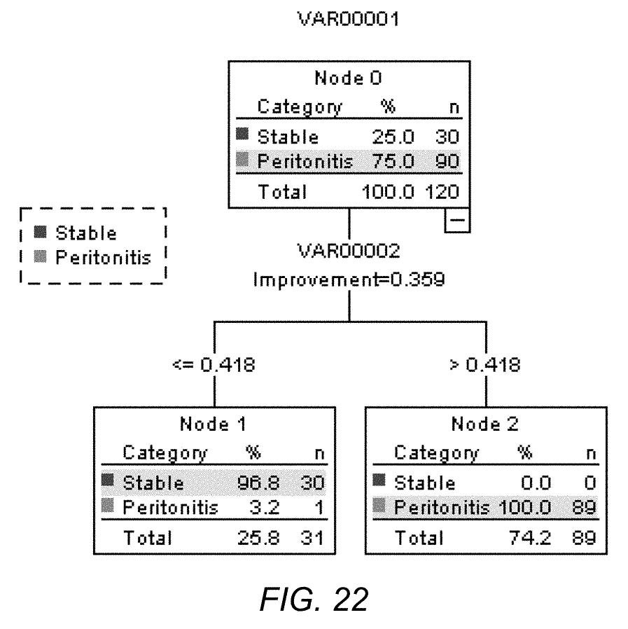

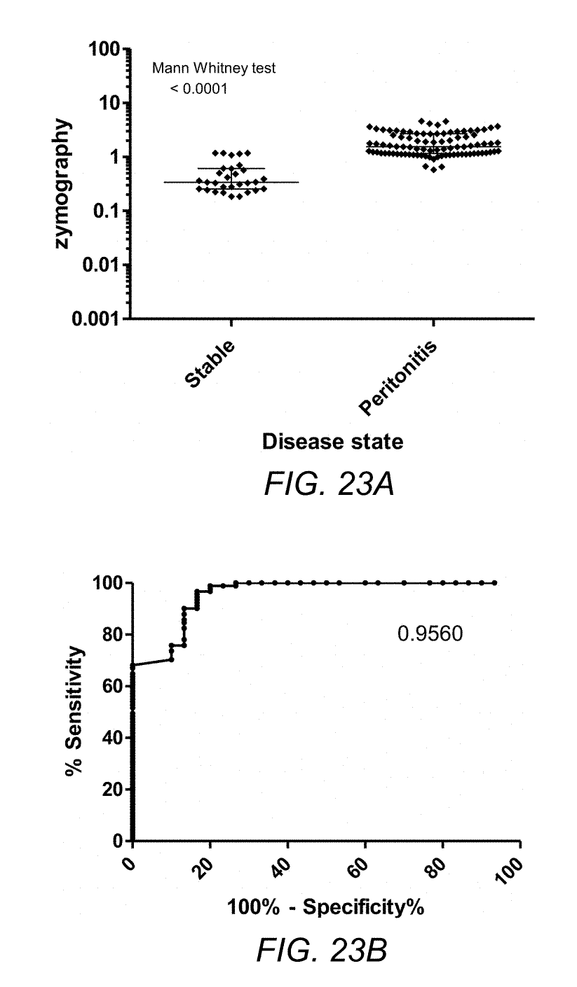

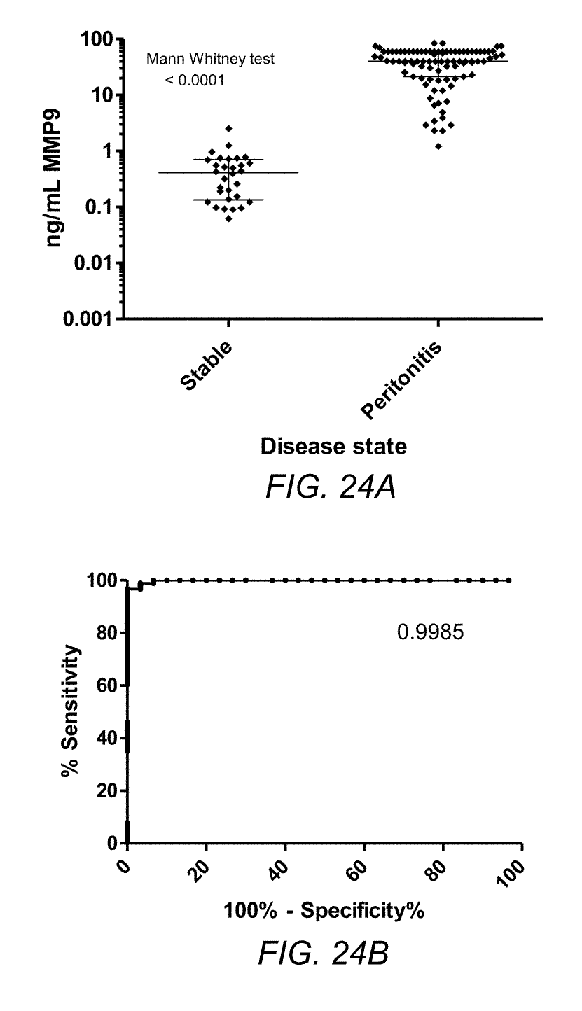

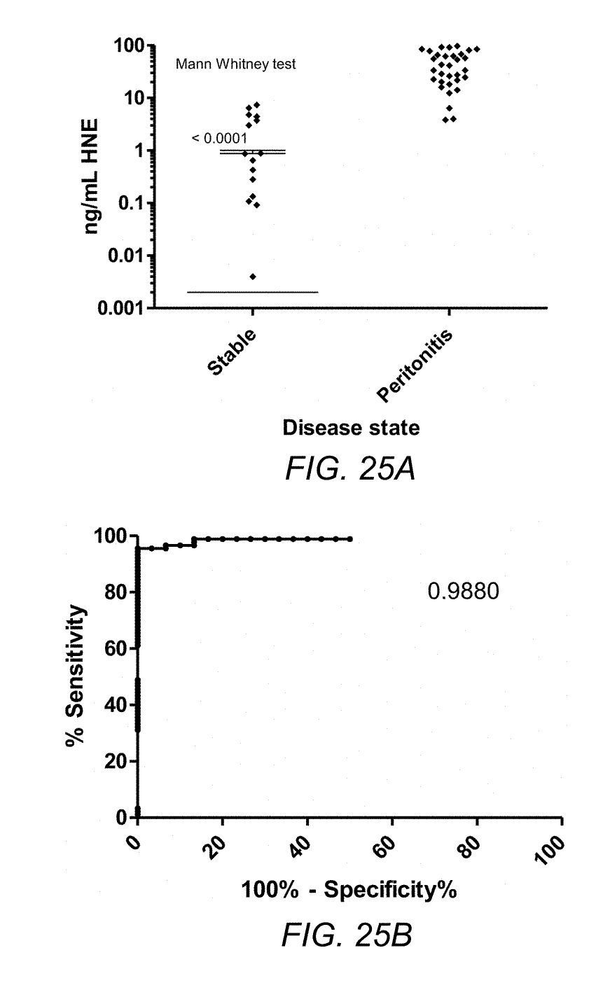

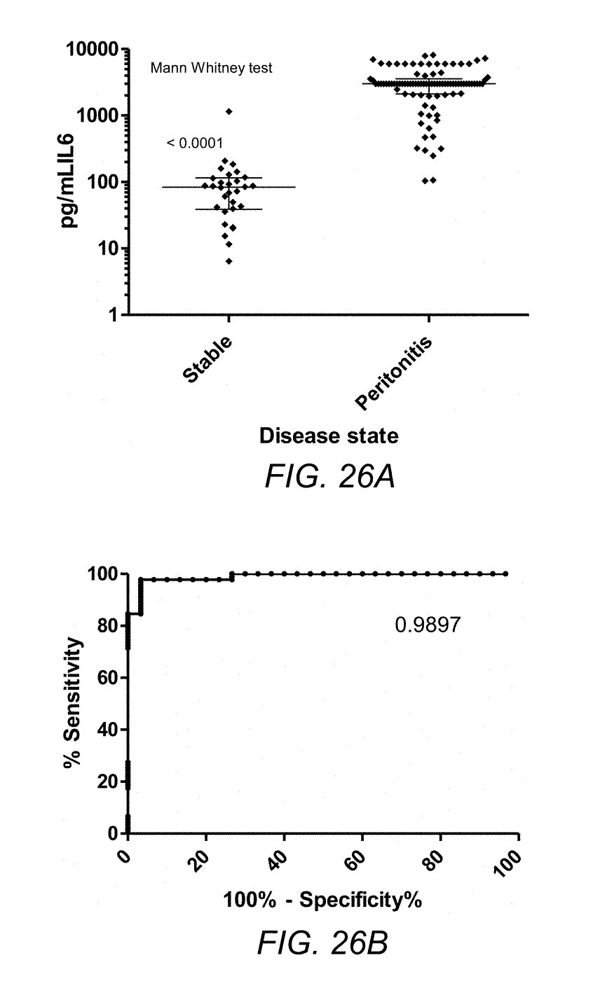

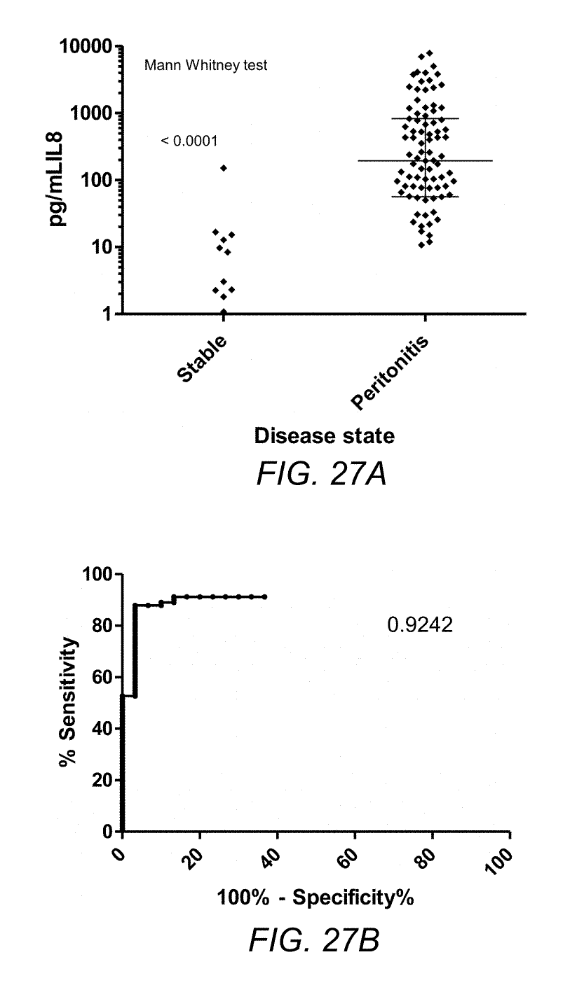

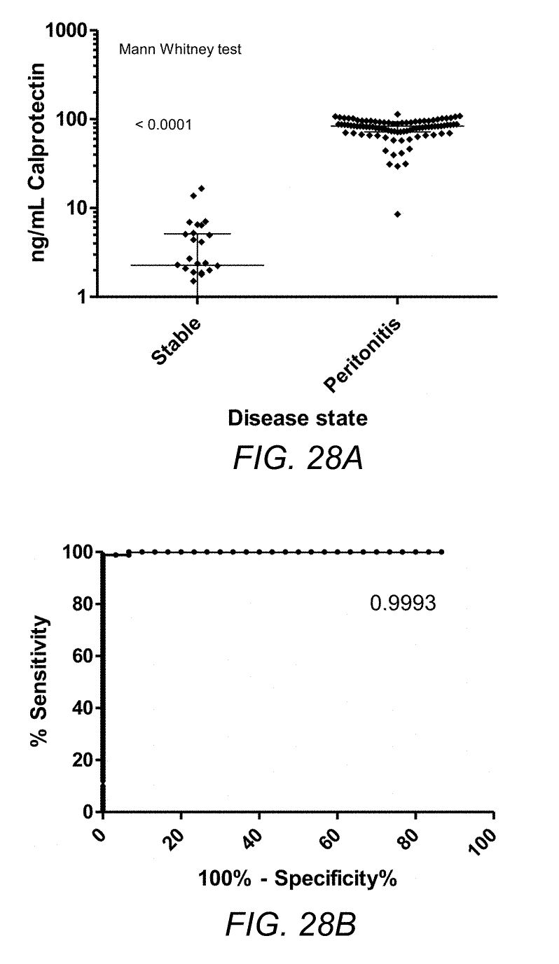

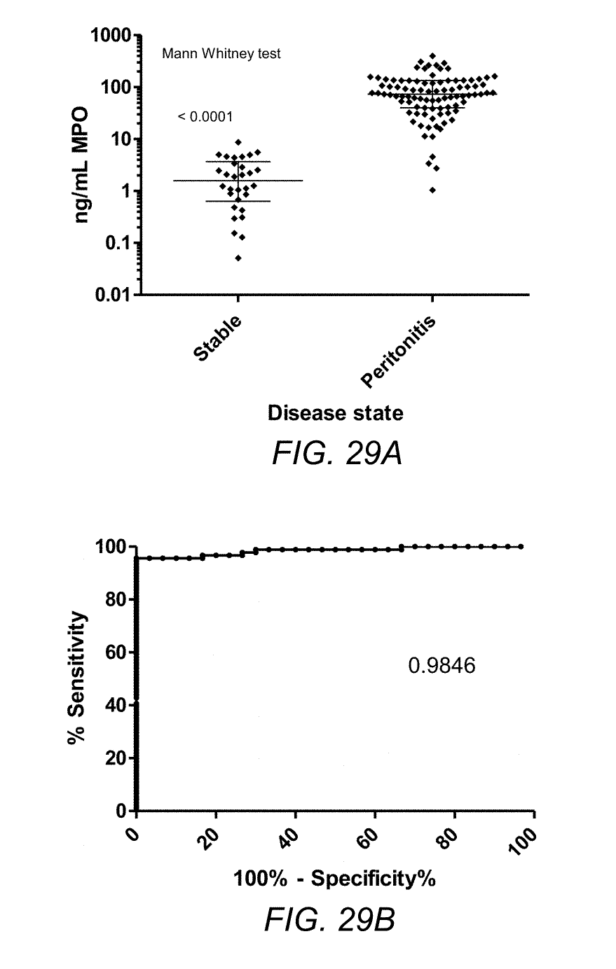

Results are shown in FIG. 22 and performance is summarised in Table 3 below:

Classification

| | Predicted | | |
|---|---|---|---|
| Observed | Stable | Peritonitis | Percent Correct |
| Stable | 30 | 0 | 100.0% |
| Peritonitis | 1 | 89 | 98.9% |
| Overall Percentage | 25.8% | 74.2% | 99.2% |

Growing Method: CRT
Dependent Variable: VAR00001

The performance of a selection of the biomarkers tested is summarised in Table 4 below:

While the above embodiments and examples have been described in the context of testing for infection of dialysate fluid, preferably contained in dialysate bags, the present disclosure is not so limited. As will be appreciated, the diagnostic test apparatus, kit of parts and methods may be applied for the testing of other bulk sources of fluid and for other purposes as set out in the appended claims.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims. Moreover, all aspects and embodiments of the invention described herein are considered to be broadly applicable and combinable with any and all other consistent embodiments, including those taken from other aspects of the invention (including in isolation) as appropriate. Various publications are cited herein, the disclosures of which are incorporated by reference in their entireties.

1.-32. (canceled)

33. A method of detecting peritonitis in a subject, selecting a subject for treatment with an antibiotic or predicting responsiveness of a subject to treatment with an antibiotic, the method comprising determining the level of at least one marker selected from MMP8, IL-6, HNE, MMP2, MMP9, TIMP1, TIMP2, NGAL, A1AT, desmosine, fibrinogen, IL-8, calprotectin, fMLP, IL1b, cystatin C, HSA, RBP4, SPD, MPO, sICAM and TNFa in a sample of peritoneal dialysate wherein:
(i) an increased level of at least one of MMP8, IL-6, HNE, MMP2, MMP9, TIMP1, TIMP2, NGAL, A1AT, desmosine, IL-8, calprotectin, fMLP, IL1b, cystatin C, HSA, RBP4, SPD, MPO, sICAM and TNFa and/or a decreased level of fibrinogen is indicative of peritonitis;
(ii) an increased level of at least one of MMP8, IL-6, HNE, MMP2, MMP9, TIMP1, TIMP2, NGAL, A1AT, desmosine, IL-8, calprotectin, fMLP, IL1b, cystatin C, HSA, RBP4, SPD, MPO, sICAM and TNFa and/or a decreased level of fibrinogen results in selection of the subject for treatment with an antibiotic; or
(iii) an increased level of at least one of MMP8, HNE, MMP2, MMP9, TIMP1, TIMP2, NGAL, A1AT, desmosine, IL-6, IL-8, calprotectin, fMLP, IL1b, cystatin C, HSA, RBP4, SPD, MPO, sICAM and TNFa and/or a decreased level of fibrinogen predicts responsiveness of the subject to treatment with an antibiotic.

34. The method of claim 33, wherein the at least one marker is selected from MMP8, HNE, MMP2, MMP9, IL-6, calprotectin and MPO.

35. A method of treating peritonitis comprising administering an antibiotic to the subject suffering from peritonitis, wherein the subject has been selected for treatment by performing the method of claim 33.

36. A method of treating peritonitis comprising administering an antibiotic to the subject suffering from peritonitis, wherein the subject displays, in a sample of peritoneal dialysate, an increased level of at least one of MMP8, IL-6, HNE, MMP2, MMP9, TIMP1, TIMP2, NGAL, A1AT, desmosine, IL-8, calprotectin, fMLP, IL1b, cystatin C, HSA, RBP4, SPD, MPO, sICAM and TNFa and/or a decreased level of fibrinogen.

37. The method of treating peritonitis of claim 35, wherein the subject displays an increased level of at least one of MMP8, HNE, MMP2, MMP9, IL-6, calprotectin and MPO.

38. The method of treating peritonitis of claim 35, wherein the antibiotic is:
a) selected from an aminoglycoside, a cephalosporin, a glycopeptide, a penicillin, a quinolone, aztreonam, clindamycin, imipenem-cilastin, linezolid, metronidazole, rifampin and an antifungal and/or
b) administered intraperitoneally, orally or intravenously (preferably intraperitoneally); and/or
c) administered during peritoneal dialysis and/or
d) administered with the dialysis fluid utilised in the peritoneal dialysis and/or
e) administered separately from the dialysis fluid utilised in the peritoneal dialysis.

39. A lateral flow testing device for detecting at least MMP8 and IL-6 in a sample, wherein the device comprises a lateral flow test strip comprising:
(a) a sample receiving zone;
(b) one or more test lines downstream of the sample receiving zone, the one or more test lines comprising immobilized or immobilizable first capture molecules which can specifically bind to MMP8 and immobilized or immobilizable second capture molecules which can specifically bind to IL-6;
(c) first reporter molecules which can specifically bind to MMP-8 specifically bound by the first capture molecules; and
(d) second reporter molecules which can specifically bind to IL-6 specifically bound by the second capture molecules;
wherein, in use, the sample is transmitted from the sample application zone to the one or more test lines resulting in capture of MMP8 and IL-6 and reporter molecules thereby detecting the markers in the sample if present.

40. The lateral flow testing device of claim 39 further comprising a control zone, downstream of the one or more test lines in relation to the sample application zone, comprising immobilized or immobilizable further capture molecules which bind to the reporter molecules or to a control molecule to indicate successful completion of an assay using the device.

41. The lateral flow testing device of claim 39, wherein the reporter molecules are labelled, either directly or indirectly.

42. The lateral flow testing device of claim 39, wherein the capture molecules and/or the reporter molecules are antibodies, or fragments or derivatives thereof.

43. A diagnostic test apparatus comprising:
an elongate housing defining a test strip holder containing a lateral flow test strip;
a fluid sampling chamber provided with an opening connecting the fluid sampling chamber with the test strip holder;
a viewing window in the elongate housing allowing reading of one or more portions of the lateral flow test strip; and
a connector configured to couple the diagnostic test apparatus to a bulk source of fluid.

44. A diagnostic test apparatus as claimed in claim 43, wherein the connector:
a) is configured to establish fluid communication between the bulk source of fluid and the fluid sampling chamber and/or
b) comprises an interference fit connector and/or
c) comprises a tubular element.

45. A diagnostic test apparatus as claimed in claim 44, wherein the tubular element:
i) contains the fluid sampling chamber and/or
ii) comprises a first end and a second end and the fluid sampling chamber is interposed between the first end and the second end.

46. A diagnostic test apparatus as claimed in claim 45, wherein the first end is open and the second end is closed to thereby enable a volume of fluid to pass into, and be retained within, the fluid sampling chamber during testing.

47. A diagnostic test apparatus as claimed in claim 44, wherein the tubular element is configured for push-fit coupling to a tube of the bulk source of fluid.

48. A diagnostic test apparatus as claimed in claim 47, wherein the tubular element comprises an O-ring seal for engaging the tube of the bulk source of fluid.

49. A diagnostic test apparatus as claimed in claim 43, wherein the connector is integral with the elongate housing.

50. A diagnostic test apparatus as claimed in claim 43, further comprising a puncturing element for opening fluid communication between the bulk source of fluid and the fluid sampling chamber.

51. A kit of parts comprising:
a diagnostic test apparatus as claimed in claim 43; and
a bulk source of fluid.

52. A method of carrying out a diagnostic test on a bulk source of fluid using a diagnostic test apparatus of the type comprising:
an elongate housing defining a test strip holder containing a lateral flow test strip, a fluid sampling chamber provided with an opening connecting the fluid sampling chamber with the test strip holder, and a viewing window in the elongate housing allowing reading of one or more portions of the lateral flow test strip;
the method comprising the steps of:
coupling the diagnostic test apparatus to the bulk source of fluid by use of a connector;
during or after coupling of the connector, establishing a fluid communication between the bulk source of fluid and the fluid sampling chamber of the diagnostic test apparatus to convey fluid from the bulk source of fluid into the fluid sampling chamber;
causing fluid to pass from the fluid sampling chamber through the opening to wet the lateral flow test strip.
